SEQUENCE LISTING (18 sequences)

SEQ ID NO: 1 - DNA, 1663 bp, homo sapiens
  CDS (122)..(1180)
agacggcaacgtggacaggaagaagcggagggcgaggaggagcagaggagcacacagatgaagcaggtgtccacgcgtccggccgtccatccgtccgtccctcctggggccggcgctgaccatgcccagcggctgccgctgcctgcatctcgtgtgcctgttgtgcattctgggggctcccggtcagcctgtccgagccgatgactgcagctcccactgtgacctggcccacggctgctgtgcacctgacggctcctgcaggtgtgacccgggctgggaggggctgcactgtgagcgctgtgtgaggatgcctggctgccagcacggtacctgccaccagccatggcagtgcatctgccacagtggctgggcaggcaagttctgtgacaaaggcttccatgggcgtgactgcgagcgcaaggctggaccctgtgaacaggcaggctccccatgccgcaatggcgggcagtgccaggacgaccagggctttgctctcaacttcacgtgccgctgcttggtgggctttgtgggtgcccgctgtgaggtaaatgtggatgactgcctgatgcggccttgtgctaacggtgccacctgccttgacggcataaaccgcttctcctgcctctgtcctgagggctttgctggacgcttctgcaccatcaacctggatgactgtgccagccgcccatgccagagaggggcccgctgtcgggaccgtgtccacgacttcgactgcctctgccccagtggctatggtggcaagacctgtgagcttgtcttacctgtcccagaccccccaaccacagtggacacccctctagggcccacctcagctgtagtggtacctgccacggggccagccccccacagcgcaggggctggtctgctgcggatctcagtgaaggaggtggtgcggaggcaagaggctgggctaggtgagcctagcttggtggccctggtggtgtttggggccctcactgctgccctggttctggctactgtgttgctgaccctgagggcctggcgccggggtgtctgcccccctggaccctgttgctaccctgccccacactatgctccagcgtgccaggaccaggagtgtcaggttagcatgctgccagcagggctccccctgccacgtgacttgccccctgagcctggaaagaccacagcactgtgatggaggtgggggctttctggcccccttcctcacctcttccacccctcagactggagtggtccgttctcaccacccttcagcttgggtacacacacagaggagacctcagcctcacaccagaaatattatttttttaatacacagaatgtaagatggaattttatcaaataaaactatgaaaatgcaagtgggctcctatgccagaaaaacccacctggcgttccagatgcaagagggccagagcagaggcctggttctggggaagcctcaggatgctgcccaccaaggagtgatttccaaagagtaatccagggtgcccttttcccttctggggaagtgtggagaggtagagccccagaggagaatgtaaacaagcagccagcacctctgtataggcccggcctggatcagagagaggggagaactctgcagggtgtgggattgggctcagggacctccgagtgaggcagggactccctgctg

SEQ ID NO: 2 - protein, 352 aa, homo sapiens
MPSGCRCLHLVCLLCILGAPGQPVRADDCSSHCDLAHGCCAPDGSCRCDPGWEGLHCERCVRMPGCQHGTCHQPWQCICHSGWAGKFCDKGFHGRDCERKAGPCEQAGSPCRNGGQCQDDQGFALNFTCRCLVGFVGARCEVNVDDCLMRPCANGATCLDGINRFSCLCPEGFAGRFCTINLDDCASRPCQRGARCRDRVHDFDCLCPSGYGGKTCELVLPVPDPPTTVDTPLGPTSAVVVPATGPAPHSAGAGLLRISVKEVVRRQEAGLGEPSLVALVVFGALTAALVLATVLLTLRAWRRGVCPPGPCCYPAPHYAPACQDQECQVSMLPAGLPLPRDLPPEPGKTTAL

SEQ ID NO: 3 - protein, 332 aa, homo sapiens
GQPVRADDCSSHCDLAHGCCAPDGSCRCDPGWEGLHCERCVRMPGCQHGTCHQPWQCICHSGWAGKFCDKGFHGRDCERKAGPCEQAGSPCRNGGQCQDDQGFALNFTCRCLVGFVGARCEVNVDDCLMRPCANGATCLDGINRFSCLCPEGFAGRFCTINLDDCASRPCQRGARCRDRVHDFDCLCPSGYGGKTCELVLPVPDPPTTVDTPLGPTSAVVVPATGPAPHSAGAGLLRISVKEVVRRQEAGLGEPSLVALVVFGALTAALVLATVLLTLRAWRRGVCPPGPCCYPAPHYAPACQDQECQVSMLPAGLPLPRDLPPEPGKTTAL

SEQ ID NO: 4 - protein, 358 aa, homo sapiens
MPSGCRCLHLVCLLCILGAPGQPVRADDCSSHCDLAHGCCAPDGSCRCDPGWEGLHCERCVRMPGCQHGTCHQPWQCICHSGWAGKFCDKGFHGRDCERKAGPCEQAGSPCRNGGQCQDDQGFALNFTCRCLVGFVGARCEVNVDDCLMRPCANGATCLDGINRFSCLCPEGFAGRFCTINLDDCASRPCQRGARCRDRVHDFDCLCPSGYGGKTCELVLPVPDPPTTVDTPLGPTSAVVVPATGPAPHSAGAGLLRISVKEVVRRQEAGLGEPSLVALVVFGALTAALVLATVLLTLRAWRRGVCPPGPCCYPAPHYAPACQDQECQVSMLPAGLPLPRDLPPEPGKTTALHHHHHH

SEQ ID NO: 5 - protein, 321 aa, homo sapiens
MPGCQHGTCHQPWQCICHSGWAGKFCDKDEHICTTQSPCQNGGQCMYDGGGEYHCVCLPGFHGRDCERKAGPCEQAGSPCRNGGQCQDDQGFALNFTCRCLVGFVGARCEVNVDDCLMRPCANGATCLDGINRFSCLCPEGFAGRFCTINLDDCASRPCQRGARCRDRVHDFDCLCPSGYGGKTCELVLPVPDPPTTVDTPLGPTSAVVVPATGPAPHSAGAGLLRISVKEVVRRQEAGLGEPSLVALVVFGALTAALVLATVLLTLRAWRRGVCPPGPCCYPAPHYAPACQDQECQVSMLPAGLPLPRDLPPEPGKTTAL

SEQ ID NO: 6 - protein, 315 aa, homo sapiens
MPGCQHGTCHQPWQCICHSGWADEHICTTQSPCQNGGQCMYDGGGEYHCVCLPGFHGRDCERKAGPCEQAGSPCRNGGQCQDDQGFALNFTCRCLVGFVGARCEVNVDDCLMRPCANGATCLDGINRFSCLCPEGFAGRFCTINLDDCASRPCQRGARCRDRVHDFDCLCPSGYGGKTCELVLPVPDPPTTVDTPLGPTSAVVVPATGPAPHSAGAGLLRISVKEVVRRQEAGLGEPSLVALVVFGALTAALVLATVLLTLRAWRRGVCPPGPCCYPAPHYAPACQDQECQVSMLPAGLPLPRDLPPEPGKTTAL

SEQ ID NO: 7 - DNA, 1149 bp, homo sapiens
  CDS (1)..(1131)
atgcccagcggctgccgctgcctgcatctcgtgtgcctgttgtgcattctgggggctcccggtcagcctgtccgagccgatgactgcagctcccactgtgacctggcccacggctgctgtgcacctgacggctcctgcaggtgtgacccgggctgggaggggctgcactgtgagcgctgtgtgaggatgcctggctgccagcacggtacctgccaccagccatggcagtgcatctgccacagtggctgggcagatgaacatatctgtaccacgcagtccccctgccagaatggaggccagtgcatgtatgacgggggcggtgagtaccattgtgtgtgcttaccaggcttccatgggcgtgactgcgagcgcaaggctggaccctgtgaacaggcaggctccccatgccgcaatggcgggcagtgccaggacgaccagggctttgctctcaacttcacgtgccgctgcttggtgggctttgtgggtgcccgctgtgaggtaaatgtggatgactgcctgatgcggccttgtgctaacggtgccacctgccttgacggcataaaccgcttctcctgcctctgtcctgagggctttgctggacgcttctgcaccatcaacctggatgactgtgccagccgcccatgccagagaggggcccgctgtcgggaccgtgtccatgacttcgactgcctctgccccagtggctatggtggcaagacttgtgagcttgtcttacctgtcccagaccccccaaccacagtggacacccctctagggcccacctcagctgtagtggtacctgccacggggccagccccccacagcgcaggggctggtctgctgcggatctcagtgaaggaggtggtgcggaggcaagaggctgggctaggtgagcctagcttggtggccctggtggtgtttggggccctcactgctgccctggttctggctactgtgttgctgaccctgagggcctggcgccggggtgtctgcccccctggaccctgttgctaccctgccccacactatgctccagcgtgccaggaccaggagtgtcaggttagcatgctgccagcagggctccccctgccacgtgacttgccccctgagcctggaaagaccacagcactgcaccatcaccatcaccat

SEQ ID NO: 8 - protein, 377 aa, homo sapiens
MPSGCRCLHLVCLLCILGAPGQPVRADDCSSHCDLAHGCCAPDGSCRCDPGWEGLHCERCVRMPGCQHGTCHQPWQCICHSGWADEHICTTQSPCQNGGQCMYDGGGEYHCVCLPGFHGRDCERKAGPCEQAGSPCRNGGQCQDDQGFALNFTCRCLVGFVGARCEVNVDDCLMRPCANGATCLDGINRFSCLCPEGFAGRFCTINLDDCASRPCQRGARCRDRVHDFDCLCPSGYGGKTCELVLPVPDPPTTVDTPLGPTSAVVVPATGPAPHSAGAGLLRISVKEVVRRQEAGLGEPSLVALVVFGALTAALVLATVLLTLRAWRRGVCPPGPCCYPAPHYAPACQDQECQVSMLPAGLPLPRDLPPEPGKTTAL

SEQ ID NO: 9 - protein, 357 aa, homo sapiens
GQPVRADDCSSHCDLAHGCCAPDGSCRCDPGWEGLHCERCVRMPGCQHGTCHQPWQCICHSGWADEHICTTQSPCQNGGQCMYDGGGEYHCVCLPGFHGRDCERKAGPCEQAGSPCRNGGQCQDDQGFALNFTCRCLVGFVGARCEVNVDDCLMRPCANGATCLDGINRFSCLCPEGFAGRFCTINLDDCASRPCQRGARCRDRVHDFDCLCPSGYGGKTCELVLPVPDPPTTVDTPLGPTSAVVVPATGPAPHSAGAGLLRISVKEVVRRQEAGLGEPSLVALVVFGALTAALVLATVLLTLRAWRRGVCPPGPCCYPAPHYAPACQDQECQVSMLPAGLPLPRDLPPEPGKTTAL

SEQ ID NO: 10 - protein, 383 aa, homo sapiens
MPSGCRCLHLVCLLCILGAPGQPVRADDCSSHCDLAHGCCAPDGSCRCDPGWEGLHCERCVRMPGCQHGTCHQPWQCICHSGWADEHICTTQSPCQNGGQCMYDGGGEYHCVCLPGFHGRDCERKAGPCEQAGSPCRNGGQCQDDQGFALNFTCRCLVGFVGARCEVNVDDCLMRPCANGATCLDGINRFSCLCPEGFAGRFCTINLDDCASRPCQRGARCRDRVHDFDCLCPSGYGGKTCELVLPVPDPPTTVDTPLGPTSAVVVPATGPAPHSAGAGLLRISVKEVVRRQEAGLGEPSLVALVVFGALTAALVLATVLLTLRAWRRGVCPPGPCCYPAPHYAPACQDQECQVSMLPAGLPLPRDLPPEPGKTTALHHHHHH

SEQ ID NO: 11 - DNA, 420 bp, homo sapiens
  CDS (1)..(402)
atgcccagcggctgccgctgcctgcatctcgtgtgcctgttgtgcattctgggggctcccggtcagcctgtccgagccgatgactgcagctcccactgtgacctggcccacggctgctgtgcacctgacggctcctgcaggtgtgacccgggctgggaggggctgcactgtgagcgctgtgtgaggatgcctggctgccagcacggtacctgccaccagccatggcagtgcatctgccacagtggctgggcaggcaagttctgtgacaaagatgaacatatctgtaccacgcagtccccctgccagaatggaggccagtgcatgtatgacgggggcggtgagtaccattgtgtgtgcttaccaggcttccatgggcgtgactgcgagcgcaaggctggaccccaccatcaccatcaccat

SEQ ID NO: 12 - protein, 134 aa, homo sapiens
MPSGCRCLHLVCLLCILGAPGQPVRADDCSSHCDLAHGCCAPDGSCRCDPGWEGLHCERCVRMPGCQHGTCHQPWQCICHSGWAGKFCDKDEHICTTQSPCQNGGQCMYDGGGEYHCVCLPGFHGRDCERKAGP

SEQ ID NO: 13 - protein, 114 aa, homo sapiens
GQPVRADDCSSHCDLAHGCCAPDGSCRCDPGWEGLHCERCVRMPGCQHGTCHQPWQCICHSGWAGKFCDKDEHICTTQSPCQNGGQCMYDGGGEYHCVCLPGFHGRDCERKAGP

SEQ ID NO: 14 - protein, 140 aa, homo sapiens
MPSGCRCLHLVCLLCILGAPGQPVRADDCSSHCDLAHGCCAPDGSCRCDPGWEGLHCERCVRMPGCQHGTCHQPWQCICHSGWAGKFCDKDEHICTTQSPCQNGGQCMYDGGGEYHCVCLPGFHGRDCERKAGPHHHHHH

SEQ ID NO: 15 - DNA, 1167 bp, homo sapiens
  CDS (1)..(1149)
atgcccagcggctgccgctgcctgcatctcgtgtgcctgttgtgcattctgggggctcccggtcagcctgtccgagccgatgactgcagctcccactgtgacctggcccacggctgctgtgcacctgacggctcctgcaggtgtgacccgggctgggaggggctgcactgtgagcgctgtgtgaggatgcctggctgccagcacggtacctgccaccagccatggcagtgcatctgccacagtggctgggcaggcaagttctgtgacaaagatgaacatatctgtaccacgcagtccccctgccagaatggaggccagtgcatgtatgacgggggcggtgagtaccattgtgtgtgcttaccaggcttccatgggcgtgactgcgagcgcaaggctggaccctgtgaacaggcaggctccccatgccgcaatggcgggcagtgccaggacgaccagggctttgctctcaacttcacgtgccgctgcttggtgggctttgtgggtgcccgctgtgaggtaaatgtggatgactgcctgatgcggccttgtgctaacggtgccacctgccttgacggcataaaccgcttctcctgcctctgtcctgagggctttgctggacgcttctgcaccatcaacctggatgactgtgccagccgcccatgccagagaggggcccgctgtcgggaccgtgtccacgacttcgactgcctctgccccagtggctatggtggcaagacctgtgagcttgtcttacctgtcccagaccccccaaccacagtggacacccctctagggcccacctcagctgtagtggtacctgccacggggccagccccccacagcgcaggggctggtctgctgcggatctcagtgaaggaggtggtgcggaggcaagaggctgggctaggtgagcctagcttggtggccctggtggtgtttggggccctcactgctgccctggttctggctactgtgttgctgaccctgagggcctggcgccggggtgtctgcccccctggaccctgttgctaccctgccccacactatgctccagcgtgccaggaccaggagtgtcaggttagcatgctgccagcagggctccccctgccacgtgacttgccccctgagcctggaaagaccacagcactgcaccatcaccatcaccat

SEQ ID NO: 16 - protein, 383 aa, homo sapiens
MPSGCRCLHLVCLLCILGAPGQPVRADDCSSHCDLAHGCCAPDGSCRCDPGWEGLHCERCVRMPGCQHGTCHQPWQCICHSGWAGKFCDKDEHICTTQSPCQNGGQCMYDGGGEYHCVCLPGFHGRDCERKAGPCEQAGSPCRNGGQCQDDQGFALNFTCRCLVGFVGARCEVNVDDCLMRPCANGATCLDGINRFSCLCPEGFAGRFCTINLDDCASRPCQRGARCRDRVHDFDCLCPSGYGGKTCELVLPVPDPPTTVDTPLGPTSAVVVPATGPAPHSAGAGLLRISVKEVVRRQEAGLGEPSLVALVVFGALTAALVLATVLLTLRAWRRGVCPPGPCCYPAPHYAPACQDQECQVSMLPAGLPLPRDLPPEPGKTTAL

SEQ ID NO: 17 - protein, 363 aa, homo sapiens
GQPVRADDCSSHCDLAHGCCAPDGSCRCDPGWEGLHCERCVRMPGCQHGTCHQPWQCICHSGWAGKFCDKDEHICTTQSPCQNGGQCMYDGGGEYHCVCLPGFHGRDCERKAGPCEQAGSPCRNGGQCQDDQGFALNFTCRCLVGFVGARCEVNVDDCLMRPCANGATCLDGINRFSCLCPEGFAGRFCTINLDDCASRPCQRGARCRDRVHDFDCLCPSGYGGKTCELVLPVPDPPTTVDTPLGPTSAVVVPATGPAPHSAGAGLLRISVKEVVRRQEAGLGEPSLVALVVFGALTAALVLATVLLTLRAWRRGVCPPGPCCYPAPHYAPACQDQECQVSMLPAGLPLPRDLPPEPGKTTAL

SEQ ID NO: 18 - protein, 389 aa, homo sapiens
MPSGCRCLHLVCLLCILGAPGQPVRADDCSSHCDLAHGCCAPDGSCRCDPGWEGLHCERCVRMPGCQHGTCHQPWQCICHSGWAGKFCDKDEHICTTQSPCQNGGQCMYDGGGEYHCVCLPGFHGRDCERKAGPCEQAGSPCRNGGQCQDDQGFALNFTCRCLVGFVGARCEVNVDDCLMRPCANGATCLDGINRFSCLCPEGFAGRFCTINLDDCASRPCQRGARCRDRVHDFDCLCPSGYGGKTCELVLPVPDPPTTVDTPLGPTSAVVVPATGPAPHSAGAGLLRISVKEVVRRQEAGLGEPSLVALVVFGALTAALVLATVLLTLRAWRRGVCPPGPCCYPAPHYAPACQDQECQVSMLPAGLPLPRDLPPEPGKTTALHHHHHH

(19) United States
(12) Patent Application Publication
(10) Pub. No.: US 2006/0155117 A1
(43) Pub. Date: Jul. 13, 2006

(54) Novel preadipocyte factor-1-like polypeptides

(75) Inventors: Jadwiga Bienkowska, Cambridge, MA (US); Gregg Mcallister, Charlestown, MA (US)

Correspondence Address: SALIWANCHIK LLOYD & SALIWANCHIK;A PROFESSIONAL ASSOCIATION, PO BOX 142950, GAINESVILLE, FL, 32614-2950, US

(73) Assignee: Applied Research Systems ARS Holding N.V., Curacao (NL)

(21) Appl. No.: 10/540,845

(22) PCT Filed: Dec. 23, 2003
(86) PCT No.: PCT/EP03/51092

§ 371 Date: Jan. 26, 2006

Related U.S. Application Data
(60) Provisional application No. 60/436,815, filed on Dec. 27, 2002.

(51) Int. Cl. C07K 14/475; C12Q 1/68; C07H 21/04; C12P 21/06; A61K 38/18

(52) U.S. Cl. 530/399; 435/6; 435/69.1; 435/320.1; 435/325; 536/23.5

(57) ABSTRACT

The present invention discloses open reading frames (ORFs) in human genome encoding for novel Preadipocyte factor-1-like polypeptides, and reagents related thereto including variants, mutants and fragments of said polypeptides, as well as ligands and antagonists directed against them. The invention provides methods for identifying and making these molecules, for preparing pharmaceutical compositions containing them, and for using them in the diagnosis, prevention and treatment of diseases.

### FIELD OF THE INVENTION

The present invention relates to nucleic acid sequences identified in human genome as encoding for novel polypeptides, more specifically for Preadipocyte factor-1-like polypeptides.

All publications, patents and patent applications cited herein are incorporated in full by reference.

### BACKGROUND OF THE INVENTION

Many novel polypeptides have been already identified by applying strict homology criteria to known polypeptides of the same family. However, since the actual content in polypeptide-encoding sequences in the human genome for Preadipocyte factor-1-like polypeptides (and for any other protein family) is still unknown, the possibility still exists to identify DNA sequence encoding polypeptide having Preadipocyte factor-1-like polypeptide activities by applying alternative and less strict homology/structural criteria to the totality of Open Reading Frames (ORFs, that is, genomic sequences containing consecutive triplets of nucleotides coding for amino acids, not interrupted by a termination codon and potentially translatable in a polypeptide) present in the human genome.

The ability for cells to make and secrete extracellular proteins is central to many biological processes. Enzymes, growth factors, extracellular matrix proteins and signalling molecules are all secreted by cells. This is through fusion of a secretory vesicle with the plasma membrane. In most cases, but not all, proteins are directed to the endoplasmic reticulum and into secretory vesicles by a signal peptide. Signal peptides are cis-acting sequences that affect the transport of polypeptide chains from the cytoplasm to a membrane bound compartment such as a secretory vesicle. Polypeptides that are targeted to the secretory vesicles are either secreted into the extracellular matrix or are retained in the plasma membrane. The polypeptides that are retained in the plasma membrane will have one or more transmembrane domains. Examples of secreted proteins that play a central role in the functioning of a cell are cytokines, hormones, extracellular matrix proteins (adhesion molecules), proteases, and growth and differentiation factors.

Preadipocyte factor-1 (Pref-1), also known as fetal antigen 1 (FA1), deltalike (dlk), or stromal cell-derived protein-1 (SCP-1) is an epidermal growth factor (EGF) repeat domain-containing transmembrane protein with an anti-adipogenic function (Smas et al (1999) J Biol. Chem. 274,12632-12641).

This protein is expressed in preadipocytes, the cells that upon receiving specific signals differentiate into mature adipocytes, but is totally absent in the mature adipocytes, the specialised cells that function to accumulate fat as stored energy. It has been found that addition of soluble Preadipocyte factor-1 to differentiating 3T3-L1 preadipocyte cultures abolishes their differentiation to adipocytes.

It is thought that there are a number of alternately spliced variants for Preadipocyte factor-1. Therefore, it is likely that Preadipocyte factor-1 acts as both an ajuxtacrine and a paracrine regulator of adipogenesis. Soluble Preadipocyte factor-1 is detectable in the circulation and is the result of post-translational modification. The EGF repeat domains of Preadipocyte factor-1 mediate cell growth and differentiation in a variety of biological settings. It is likely that these domain are involved in binding to putative Preadipocyte factor-1 receptor (s) on cells in order to maintain the preadipose phenotype.

In developing embryos, Preadipocyte factor-1 is expressed in numerous tissues including pituitary, liver, lung, tongue and vertebrae, indicating that the expression of Preadipocyte factor-1 may be involved in preventing terminal differentiation and allowing cell proliferation. In adults, Preadipocyte factor-1 expression is critical for the adrenal cortical zona glomerulosa differentiation (Raza et al (1998) Endocr. Res. 24,977-81). Failure of the zona glomerulosa to differentiate can lead to increased resorption of sodium, increased resorption of water, with consequent expansion of extracellular fluid volume and increased renal excretion of potassium.

Thus, dysregulation of Preadipocyte factor-1 expression may lead to various conditions such as obesity, organomegaly, diabetes (including insulin resistance, hyperinsulinemia, hyperglycemia, hypertriglyceridemia), adrenocortical dysfunction, hypertrophy of cardiac and skeletal muscle, lipodystrophy, and immune system disorders including autoimmune diseases and immunodeficiencies, developmental defects, cancer, autoimmune thyroid diseases and related disorders such as opthalmopathies and other pathological conditions. Particularly for this reason, Preadipocyte factor 1, and molecules related thereto are of significant interest in increasing understanding of the underlying pathways that lead to the various disease states in which these proteins are implicated, and in developing more effective gene or drug therapies to treat these disorders.

### SUMMARY OF THE INVENTION

The invention is based upon the identification of an Open Reading Frame (ORF) in the human genome encoding a novel Preadipocyte factor-1-like polypeptide. This polypeptide will be referred to herein as the SCS0009 polypeptide. Based on the SCS0009 polypeptide, five other splice variants were identified: SCS0009-SV1, SCS0009-SV2, SCS0009-SV3, SCS0009-SV4 and SCS0009-SV5, of which SCS0009-SV3 has not been described before.

Accordingly, the invention provides isolated SCS0009 polypeptides having the amino acid sequence given by SEQ ID NO: 2, SEQ ID NO: 8, their mature forms, their histidine tagged forms, variants, and fragments, as polypeptides having the activity of Preadipocyte factor-1-like polypeptides. The invention includes also the nucleic acids encoding them, vectors containing such nucleic acids, and cell containing these vectors or nucleic acids, as well as other related reagents such as fusion proteins, ligands, and antagonists.

The invention provides methods for identifying and making these molecules, for preparing pharmaceutical compositions containing them, and for using them in the diagnosis, prevention and treatment of diseases.

### BRIEF DESCRIPTION OF THE FIGURES

FIG. 1: alignment of the SCS0009 ORF with known related polypeptide sequences.

FIG. 2: Clustal W alignment of predicted amino acid sequences of SCS0009 prediction and splice variants SV3, SV4 and SV5.

FIG. 3: Clustal W alignment of predicted amino acid sequences of SCS0009 with SV1 and SV2.

FIG. 4: Nucleotide sequence of SCS0009 prediction with translation.

FIG. 5: Nucleotide sequence with translation of cDNA Insert In Image clone 5478078 (SCS0009-SV3).

FIG. 6: Alignment of predicted amino acid sequence of SCS0009 with SCS0009-SV3.

FIG. 7: Map of expression vector pEAK12d.

FIG. 8: Map of Expression vector pDEST12.2.

FIG. 9: Map of pDONR 221.

FIG. 10: Map of pENTR-SCS0009SV3-6HIS.

FIG. 11: Map of pEAK12d-SCS0009SV3-6HIS.

FIG. 12: Map of pDEST12.2-SCS0009SV3-6HIS.

FIG. 13: Nucleotide sequence of SCS0009 prediction with translation.

FIG. 14: Nucleotide sequence with translation of cDNA insert in image clone 3349698 (SCS0009-SV4).

FIG. 15: Alignment of predicted amino acid sequence of SCS0009 with SCS0009-SV4.

FIG. 16: Map of expression vector pEAK12d.

FIG. 17: Map of Expression vector pDEST12.2.

FIG. 18: Map of pDONR 221.

FIG. 19: Map of pENTR-SCS0009SV4-6HIS.

FIG. 20: Map of pEAK12d-SCS0009SV4-6HIS.

FIG. 21: Map of pDEST12.2-SCS0009SV4-6HIS.

FIG. 22: Nucleotide sequence of SCS0009 prediction with translation.

FIG. 23: Nucleotide sequence with translation of SCS0009 -SV5 PCR product indicating the positions of the SCS0009-AP1, -AP2, -AP3 and -AP4 primers used to generate the SCS0009 sequence.

FIG. 24: Nucleotide sequence and translation of cloned SCS0009 ORF.

FIG. 25: Map of pCR-BluntII-TOPO-SCS0009.

FIG. 26: Map of pDONR 221.

FIG. 27: Map of expression vector pEAK12d.

FIG. 28: Map of Expression vector pDEST12.2.

FIG. 29: Map of pENTR-SCS0009-6HIS.

FIG. 30: Map of pEAK12d-SCS0009-6HIS.

FIG. 31: Map of pDEST12.2-SCS0009-6HIS.

FIG. 32. SMART Domains alignment of the SCS0009 polypeptides.

### DETAILED DESCRIPTION OF THE INVENTION

According to a first aspect of the present invention there is provided an isolated polypeptide having Preadipocyte factor-1-like activity selected from the group consisting of:
a) the amino acid sequences as recited in SEQ ID NO: 2 or SEQ ID NO: 8;
b) the mature form of the polypeptides whose sequence are recited in SEQ ID NO: 2 (SEQ ID NO:3) or SEQ ID NO: 8 (SEQ ID NO:9);
c) the histidine tagged form of the polypeptides whose sequence are recited in SEQ ID NO: 2 (SEQ ID NO:4) or SEQ ID NO: 8 (SEQ ID NO:10);
d) a variant of the amino acid sequences recited in SEQ ID NO: 2 or SEQ ID NO: 8, wherein any amino acid specified in the chosen sequence is non-conservatively substituted, provided that no more than 15% of the amino acid residues in the sequence are so changed;
e) an active fragment, precursor, salt, or derivative of the amino acid sequences given in a) to d).

The novel polypeptide described herein was identified on the basis of a consensus sequence for human Preadipocyte factor-1-like polypeptides in which the number and the positioning of selected amino acids are defined for a protein sequence having a length comparable to known Preadipocyte factor-1 -like polypeptides.

The totality of amino acid sequences obtained by translating the known ORFs in the human genome were challenged using this consensus sequence, and the positive hits were further screened for the presence of predicted specific structural and functional “signatures” that are distinctive of a polypeptide of this nature, and finally selected by comparing sequence features with known Preadipocyte factor-1-like polypeptides. Therefore, the novel polypeptides of the invention can be predicted to have Preadipocyte factor-1-like activities.

The terms “active” and “activity” refer to the Preadipocyte factor-1-like properties predicted for the Preadipocyte factor-1-like polypeptide whose amino acid sequence is presented in SEQ ID NO: 2, SEQ ID NO: 3, SEQ ID NO: 4, SEQ ID NO: 8, SEQ ID NO: 9 or SEQ ID NO: 10 in the present application. These properties include the ability to prevent terminal differentiation of preadipocytes and allow cell proliferation.

In a second aspect, the invention provides a purified nucleic acid molecule which encodes a polypeptide of the first aspect of the invention. Preferably, the purified nucleic acid molecule comprises or consists of the nucleic acid sequences as recited in SEQ ID NO:1 or SEQ ID NO:7 (encoding the Preadipocyte factor-1-like polypeptides whose amino acid sequences are recited in SEQ ID NO:2 or SEQ ID NO:8), particularly the coding sequence which starts at nucleotide 122 and ends at 1180 in SEQ ID NO:1 or starts at nucleotide 1 and ends at 1131 in SEQ ID NO:8.

In a third aspect, the invention provides a purified nucleic acid molecule which hydridizes under high stringency conditions with a nucleic acid molecule of the second aspect of the invention.

In a fourth aspect, the invention provides a vector, such as an expression vector, that contains a nucleic acid molecule of the second or third aspect of the invention.

In a fifth aspect, the invention provides a host cell transformed with a vector of the fourth aspect of the invention.

In a sixth aspect, the invention provides a ligand which binds specifically to, and which preferably inhibits the metalloprotease activity of a polypeptide of the first aspect of the invention. Ligands to a polypeptide according to the invention may come in various forms, including natural or modified substrates, enzymes, receptors, small organic molecules such as small natural or synthetic organic molecules of up to 2000 Da, preferably 800 Da or less, peptidomimetics, inorganic molecules, peptides, polypeptides, antibodies, structural or functional mimetics of the aforementioned.

In a seventh aspect, the invention provides a compound that is effective to alter the expression of a natural gene which encodes a polypeptide of the first aspect of the invention or to regulate the activity of a polypeptide of the first aspect of the invention.

A compound of the seventh aspect of the invention may either increase (agonise) or decrease (antagonise) the level of expression of the gene or the activity of the polypeptide. Importantly, the identification of the function of the Preadipocyte factor-1-like polypeptide of the invention allows for the design of screening methods capable of identifying compounds that are effective in the treatment and/or diagnosis of disease.

In an eighth aspect, the invention provides a polypeptide of the first aspect of the invention, or a nucleic acid molecule of the second or third aspect of the invention, or a vector of the fourth aspect of the invention, or a host cell of the fifth aspect of the invention, or a ligand of the sixth aspect of the invention, or a compound of the seventh aspect of the invention, for use in therapy or diagnosis. These molecules may also be used in the manufacture of a medicament for the treatment of obesity, organomegaly, diabetes (including insulin resistance, hyperinsulinemia, hyperglycemia, hypertriglyceridemia), adrenocortical dysfunction, hypertrophy of cardiac and skeletal muscle, lipodystrophy, and immune system disorders including autoimmune diseases and immunodeficiencies, developmental defects, cancer, autoimmune thyroid diseases and related disorders such as opthalmopathies and other pathological conditions.

In a ninth aspect, the invention provides a method of diagnosing a disease in a patient, comprising assessing the level of expression of a natural gene encoding a polypeptide of the first aspect of the invention or the activity of a polypeptide of the first aspect of the invention in tissue from said patient and comparing said level of expression or activity to a control level, wherein a level that is different to said control level is indicative of disease. Such a method will preferably be carried out in vitro. Similar methods may be used for monitoring the therapeutic treatment of disease in a patient, wherein altering the level of expression or activity of a polypeptide or nucleic acid molecule over the period of time towards a control level is indicative of regression of disease.

A preferred method for detecting polypeptides of the first aspect of the invention comprises the steps of: (a) contacting a ligand, such as an antibody, of the sixth aspect of the invention with a biological sample under conditions suitable for the formation of a ligand-polypeptide complex; and (b) detecting said complex.

A number of different such methods according to the ninth aspect of the invention exist, as the skilled reader will be aware, such as methods of nucleic acid hybridization with short probes, point mutation analysis, polymerase chain reaction (PCR) amplification and methods using antibodies to detect aberrant protein levels. Similar methods may be used on a short or long term basis to allow therapeutic treatment of a disease to be monitored in a patient. The invention also provides kits that are useful in these methods for diagnosing disease.

In a tenth aspect, the invention provides for the use of a polypeptide of the first aspect of the invention as a Preadipocyte factor-1-like protein. Suitable uses include use as a secreted glycoprotein, in particular in the context of preventing terminal differentiation of preadipocytes and allowing cell proliferation.

In an eleventh aspect, the invention provides a pharmaceutical composition comprising a polypeptide of the first aspect of the invention, or a nucleic acid molecule of the second or third aspect of the invention, or a vector of the fourth aspect of the invention, or a host cell of the fifth aspect of the invention, or a ligand of the sixth aspect of the invention, or a compound of the seventh aspect of the invention, in conjunction with a pharmaceutically-acceptable carrier.

In a twelfth aspect, the present invention provides a polypeptide of the first aspect of the invention, or a nucleic acid molecule of the second or third aspect of the invention, or a vector of the fourth aspect of the invention, or a host cell of the fifth aspect of the invention, or a ligand of the sixth aspect of the invention, or a compound of the seventh aspect of the invention, for use in the manufacture of a medicament for the diagnosis or treatment of a disease, such as for the treatment of prevention of obesity, organomegaly, diabetes (including insulin resistance, hyperinsulinemia, hyperglycemia, hypertriglyceridemia), adrenocortical dysfunction, hypertrophy of cardiac and skeletal muscle, lipodystrophy, and immune system disorders including autoimmune diseases and immunodeficiencies, developmental defects, cancer, autoimmune thyroid diseases and related disorders such as opthalmopathies and other pathological conditions.

In a thirteenth aspect, the invention provides a method of treating a disease in a patient comprising administering to the patient a polypeptide of the first aspect of the invention, or a nucleic acid molecule of the second or third aspect of the invention, or a vector of the fourth aspect of the invention, or a host cell of the fifth aspect of the invention, or a ligand of the sixth aspect of the invention, or a compound of the seventh aspect of the invention.

For diseases In which the expression of a natural gene encoding a polypeptide of the first aspect of the invention, or in which the activity of a polypeptide of the first aspect of the invention, is lower in a diseased patient when compared to the level of expression or activity in a healthy patient, the polypeptide, nucleic acid molecule, ligand or compound administered to the patient should be an agonist. Conversely, for diseases in which the expression of the natural gene or activity of the polypeptide is higher in a diseased patient when compared to the level of expression or activity in a healthy patient, the polypeptide, nucleic acid molecule, ligand or compound administered to the patient should be an antagonist. Examples of such antagonists include antisense nucleic acid molecules, ribozymes and ligands, such as antibodies.

In a fourteenth aspect, the invention provides transgenic or knockout non-human animals that have been transformed to express higher, lower or absent levels of a polypeptide of the first aspect of the invention. Such transgenic animals are very useful models for the study of disease and may also be used in screening regimes for the identification of compounds that are effective in the treatment or diagnosis of such a disease.

A summary of standard techniques and procedures which may be employed in order to utilise the invention is given below. It will be understood that this invention is not limited to the particular methodology, protocols, cell lines, vectors and reagents described. It is also to be understood that the terminology used herein is for the purpose of describing particular embodiments only and it is not intended that this terminology should limit the scope of the present invention. The extent of the invention is limited only by the terms of the appended claims.

Standard abbreviations for nucleotides and amino acids are used in this specification.

The practice of the present invention will employ, unless otherwise indicated, conventional techniques of molecular biology, microbiology, recombinant DNA technology and immunology, which are within the skill of the those working in the art

Such techniques are explained fully in the literature. Examples of particularly suitable texts for consultation include the following: Sambrook Molecular Cloning; A Laboratory Manual, Second Edition (1989); DNA Cloning, Volumes I and II (D. N Glover ed. 1985); Oligonucleotide Synthesis (M. J. Gait ed. 1984); Nucleic Acid Hybridization (B. D. Hames & S. J. Higgins eds. 1984); Transcription and Translation (B. D. Hames & S. J. Higgins eds. 1984); Animal Cell Culture (R. I. Freshney ed. 1986); Immobilized Cells and Enzymes (IRL Press, 1986); B. Perbal, A Practical Guide to Molecular Cloning (1984); the Methods in Enzymology series (Academic Press, Inc.), especially volumes 154 & 155; Gene Transfer Vectors for Mammalian Cells (J. H. Miller and M. P. Calos eds. 1987, Cold Spring Harbor Laboratory); Immunochemical Methods. in Cell and Molecular Biology (Mayer and Walker, eds. 1987, Academic Press, London); Scopes, (1987) Protein Purification: Principles and Practice, Second Edition (Springer Verlag, N.Y.); and Handbook of Experimental Immunology, Volumes I-IV (D. M. Weir and C. C. Blackwell eds. 1986).

The first aspect of the invention includes variants of the amino acid sequence recited in SEQ ID NO: 2, SEQ ID NO: 3, SEQ ID NO: 4, SEQ ID NO: 8, SEQ ID NO: 9 or SEQ ID NO: 10 wherein any amino acid specified in the chosen sequence is non-conservatively substituted, provided that no more than 15% of the amino acid residues in the sequence are so changed. Protein sequences having the indicated number of non-conservative substitutions can be identified using commonly available bioinformatic tools (Mulder N J and Apweiler R, 2002; Rehm B H, 2001).

In addition to such sequences, a series of polypeptides forms part of the disclosure of the invention. Being Preadipocyte factor-1-like polypeptides known to go through maturation processes including the proteolytic removal of N-terminal sequences (by signal peptidases and other proteolytic enzymes), the present application also claims the mature form of the polypeptide whose sequence is recited in SEQ ID NO: 2 (SEQ ID NO: 4) or SEQ ID NO: 8 (SEQ ID NO: 9). Mature forms are intended to include any polypeptide showing Preadipocyte factor-1-like activity and resulting from in vivo (by the expressing cells or animals) or in vitro (by modifying the purified polypeptides with specific enzymes) post-translational maturation processes. Other alternative mature forms can also result from the addition of chemical groups such as sugars or phosphates. The present application also claims the histidine tagged forms of the polypeptides whose sequences are recited in SEQ ID NO: 2 (SEQ ID NO:4) or SEQ ID NO: 8 (SEQ ID NO:10).

Other claimed polypeptides are the active variants of the amino acid sequences given by SEQ ID NO: 2, SEQ ID NO: 3, SEQ ID NO: 4, SEQ ID NO: 8, SEQ ID NO: 9 or SEQ ID NO: 10 wherein any amino acid specified in the chosen sequence is non-conservatively substituted, provided that no more than 15%, preferably no more that 10%, 5%, 3%, or 1%, of the amino acid residues in the sequence are so changed. The indicated percentage has to be measured over the novel amino acid sequences disclosed.

In accordance with the present invention, any substitution should be preferably a “conservative” or “safe” substitution, which is commonly defined a substitution introducing an amino acids having sufficiently similar chemical properties (eg a basic, positively charged amino acid should be replaced by another basic, positively charged amino acid), in order to preserve the structure and the biological function of the molecule.

The literature provide many models on which the selection of conservative amino acids substitutions can be performed on the basis of statistical and physico-chemical studies on the sequence and/or the structure of proteins (Rogov S I and Nekrasov A N, 2001). Protein design experiments have shown that the use of specific subsets of amino acids can produce foldable and active proteins, helping in the classification of amino acid “synonymous” substitutions which can be more easily accommodated in protein structure, and which can be used to detect functional and structural homologs and paralogs (Murphy L R et al., 2000). The groups of synonymous amino acids and the groups of more preferred synonymous amino acids are shown in Table I.

Active variants having comparable, or even improved, activity with respect of corresponding Preadipocyte factor-1-like polypeptides may result from conventional mutagenesis technique of the encoding DNA, from combinatorial technologies at the level of encoding DNA sequence (such as DNA shuffling, phage display/selection), or from computer-aided design studies, followed by the validation for the desired activities as described in the prior art.

Specific, non-conservative mutations can be also introduced in the polypeptides of the invention with different purposes. Mutations reducing the affinity of the Preadipocyte factor-1-like polypeptide may increase its ability to be reused and recycled, potentially increasing its therapeutic potency (Robinson C R, 2002). Immunogenic epitopes eventually present in the polypeptides of the invention can be exploited for developing vaccines (Stevanovic S, 2002), or eliminated by modifying their sequence following known methods for selecting mutations for increasing protein stability, and correcting them (van den Burg B and Eijsink V, 2002; WO 02/05146, WO 00/34317, WO 98/52976).

Further alternative polypeptides of the invention are active fragments, precursors, salts, or functionally-equivalent derivatives of the amino acid sequences described above.

Fragments should present deletions of terminal or internal amino acids not altering their function, and should involve generally a few amino acids, e.g., under ten, and preferably under three, without removing or displacing amino acids which are critical to the functional conformation of the proteins. Small fragments may form an antigenic determinant.

The “precursors” are compounds which can be converted into the compounds of present invention by metabolic and enzymatic processing prior or after the administration to the cells or to the body.

The term “salts” herein refers to both salts of carboxyl groups and to acid addition salts of amino groups of the polypeptides of the present invention. Salts of a carboxyl group may be formed by means known in the art and include inorganic salts, for example, sodium, calcium, ammonium, ferric or zinc salts, and the like, and salts with organic bases as those formed, for example, with amines, such as triethanolamine, arginine or lysine, piperidine, procaine and the like. Acid addition salts include, for example, salts with mineral acids such as, for example, hydrochloric acid or sulfuric acid, and salts with organic acids such as, for example, acetic acid or oxalic acid. Any of such salts should have substantially similar activity to the peptides and polypeptides of the invention or their analogs.

The term “derivatives” as herein used refers to derivatives which can be prepared from the functional groups present on the lateral chains of the amino acid moieties or on the amino- or carboxy-terminal groups according to known methods. Such molecules can result also from other modifications which do not normally alter primary sequence, for example in vivo or in vitro chemical derivativization of polypeptides (acetylation or carboxylation), those made by modifying the pattern of phosphorylation (introduction of phosphotyrosine, phosphoserine, or phosphothreonine residues) or glycosylation (by exposing the polypeptide to mammalian glycosylating enzymes) of a peptide during its synthesis and processing or in further processing steps. Alternatively, derivatives may include esters or aliphatic amides of the carboxyl-groups and N-acyl derivatives of free amino groups or O-acyl derivatives of free hydroxyl-groups and are formed with acyl-groups as for example alcanoyl- or aryl-groups.

The generation of the derivatives may involve a site-directed modification of an appropriate residue, in an internal or terminal position. The residues used for attachment should they have a side-chain amenable for polymer attachment (i.e., the side chain of an amino acid bearing a functional group, e.g., lysine, aspartic acid, glutamic acid, cysteine, histidine, etc.). Alternatively, a residue having a side chain amenable for polymer attachment can replace an amino acid of the polypeptide, or can be added in an internal or terminal position of the polypeptide. Also, the side chains of the genetically encoded amino acids can be chemically modified for polymer attachment, or unnatural amino acids with appropriate side chain functional groups can be employed. The preferred method of attachment employs a combination of peptide synthesis and chemical ligation. Advantageously, the attachment of a water-soluble polymer will be through a biodegradable linker, especially at the amino-terminal region of a protein. Such modification acts to provide the protein in a precursor (or “pro-drug”) form, that, upon degradation of the linker releases the protein without polymer modification.

Polymer attachment may be not only to the side chain of the amino acid naturally occurring in a specific position of the antagonist or to the side chain of a natural or unnatural amino acid that replaces the amino acid naturally occurring in a specific position of the antagonist, but also to a carbohydrate or other moiety that is attached to the side chain of the amino acid at the target position. Rare or unnatural amino acids can be also introduced by expressing the protein in specifically engineered bacterial strains (Bock A, 2001).

All the above indicated variants can be natural, being identified in organisms other than humans, or artificial, being prepared by chemical synthesis, by site-directed mutagenesis techniques, or any other known technique suitable thereof, which provide a finite set of substantially corresponding mutated or shortened peptides or polypeptides which can be routinely obtained and tested by one of ordinary skill in the art using the teachings presented in the prior art.

The novel amino acid sequences disclosed in the present patent application can be used to provide different kind of reagents and molecules. Examples of these compounds are binding proteins or antibodies that can be identified using their full sequence or specific fragments, such as antigenic determinants. Peptide libraries can be used in known methods (Tribbick G, 2002) for screening and characterizing antibodies or other proteins binding the claimed amino acid sequences, and for identifying alternative forms of the polypeptides of the invention having similar binding properties.

The present patent application discloses also fusion proteins comprising any of the polypeptides described above. These polypeptides should contain protein sequence heterologous to the one disclosed in the present patent application, without significantly impairing the Preadipocyte factor-1-like activity of the polypeptide and possibly providing additional properties. Examples of such properties are an easier purification procedure, a longer lasting half-life in body fluids, an additional binding moiety, the maturation by means of an endoproteolytic digestion, or extracellular localization. This latter feature is of particular importance for defining a specific group of fusion or chimeric proteins included in the above definition since it allows the claimed molecules to be localized in the space where not only isolation and purification of these polypeptides is facilitated, but also where generally Preadipocyte factor-1-like polypeptides and their receptor interact.

Design of the moieties, ligands, and linkers, as well methods and strategies for the construction, purification, detection and use of fusion proteins are disclosed in the literature (Nilsson J et al., 1997; Methods Enzymol, Vol. 326-328, Academic Press, 2000). The preferred one or more protein sequences which can be comprised in the fusion proteins belong to these protein sequences: membrane-bound protein, immunoglobulin constant region, multimerization domains, extracellular proteins, signal peptide-containing proteins, export signal-containing proteins. Features of these sequences and their specific uses are disclosed in a detailed manner, for example, for albumin fusion proteins (WO 01/77137), fusion proteins including multimerization domain (WO 01/02440, WO 00/24782), immunoconjugates (Garnett M C, 2001), or fusion protein providing additional sequences which can be used for purifying the recombinant products by affinity chromatography (Constans A, 2002; Burgess R R and Thompson N E, 2002; Lowe C R et al., 2001; J. Bioch. Biophy. Meth., vol. 49 (1-3), 2001; Sheibani N, 1999).

The polypeptides of the invention can be used to generate and characterize ligands binding specifically to them. These molecules can be natural or artificial, very different from the chemical point of view (binding proteins, antibodies, molecularly imprinted polymers), and can be produced by applying the teachings in the art (WO 02/74938; Kuroiwa Y et al, 2002; Haupt K, 2002; van Dijk M A and van de Winkel J G, 2001; Gavilondo J V and Larrick J W, 2000). Such ligands can antagonize or inhibit the Preadipocyte factor-1-like activity of the polypeptide against which they have been generated. In particular, common and efficient ligands are represented by extracellular domain of a membrane-bound protein or antibodies, which can be in the form monoclonal, polyclonal, humanized antibody, or an antigen binding fragment.

The polypeptides and the polypeptide-based derived reagents described above can be in alternative forms, according to the desired method of use and/or production, such as active conjugates or complexes with a molecule chosen amongst radioactive labels, fluorescent labels, biotin, or cytotoxic agents.

Specific molecules, such as peptide mimetics, can be also designed on the sequence and/or the structure of a polypeptide of the invention. Peptide mimetics (also called peptidomimetics) are peptides chemically modified at the level of amino acid side chains, of amino acid chirality, and/or of the peptide backbone. These alterations are intended to provide agonists or antagonists of the polypeptides of the invention with improved preparation, potency and/or pharmacokinetics features.

For example, when the peptide is susceptible to cleavage by peptidases following injection into the subject is a problem, replacement of a particularly sensitive peptide bond with a non-cleavable peptide mimetic can provide a peptide more stable and thus more useful as a therapeutic. Similarly, the replacement of an L-amino acid residue is a standard way of rendering the peptide less sensitive to proteolysis, and finally more similar to organic compounds other than peptides. Also useful are amino-terminal blocking groups such as t-butyloxycarbonyl, acetyl, theyl, succinyl, methoxysuccinyl, suberyl, adipyl, azelayl, dansyl, benzyloxycarbonyl, fluorenylmethoxycarbonyl, methoxyazelayl, methoxyadipyl, methoxysuberyl, and 2,4-dinitrophenyl. Many other modifications providing increased potency, prolonged activity, easiness of purification, and/or increased half-life are disclosed in the prior art (WO 02/10195; Villain M et al., 2001).

Preferred alternative, synonymous groups for amino acids derivatives included in peptide mimetics are those defined in Table II. A non-exhaustive list of amino acid derivatives also include aminoisobutyric acid (Aib), hydroxyproline (Hyp), 1,2,3,4-tetrahydro-isoquinoline-3-COOH, indoline-2carboxylic acid, 4-difluoro-proline, L-thiazolidine-4-carboxylic acid, L-homoproline, 3,4-dehydro-proline, 3,4-dihydroxy-phenylalanine, cyclohexyl-glycine, and phenylglycine.

By “amino acid derivative” is intended an amino acid or amino acid-like chemical entity other than one of the 20 genetically encoded naturally occurring amino acids. In particular, the amino acid derivative may contain substituted or non-substituted, linear, branched, or cyclic alkyl moieties, and may include one or more heteroatoms. The amino acid derivatives can be made de novo or obtained from commercial sources (Calbiochem-Novabiochem AG, Switzerland; Bachem, USA).

Various methodologies for incorporating unnatural amino acids derivatives into proteins, using both in vitro and in vivo translation systems, to probe and/or improve protein structure and function are disclosed in the literature (Dougherty D A, 2000). Techniques for the synthesis and the development of peptide mimetics, as well as non-peptide mimetics, are also well known in the art (Golebiowski A et al., 2001; Hruby V J and Balse P M, 2000; Sawyer T K, in “Structure Based Drug Design”, edited by Veerapandian P, Marcel Dekker Inc., pg. 557-663, 1997).

Another object of the present invention are isolated nucleic acids encoding for the polypeptides of the invention having Preadipocyte factor-1-like activity, the polypeptides binding to an antibody or a binding protein generated against them, the corresponding fusion proteins, or mutants having antagonistic activity as disclosed above. Preferably, these nucleic acids should comprise a DNA sequence selected from the group consisting of SEQ ID NO: 1, a polynucleotide defined by nucleotides 122 to 1180 of SEQ ID NO:1, of SEQ ID NO: 7, a polynucleotide defined by nucleotides 1 to 1131 of SEQ ID NO:7, or the complement of either of said DNA sequences.

Alternatively, the nucleic acids of the invention should hybridize under high stringency conditions, or exhibit at least about 85% identity over a stretch of at least about 30 nucleotides, with a nucleic acid consisting of SEQ ID NO: 1, a polynucleotide defined by nucleotides 122 to 1180 of SEQ ID NO:1, of SEQ ID NO: 7, a polynucleotide defined by nucleotides 1 to 1131 of SEQ ID NO:7, or the complement of either of said DNA sequences.

The wording “high stringency conditions” refers to conditions in a hybridization reaction that facilitate the association of very similar molecules and consist in the overnight incubation at 60-65° C. in a solution comprising 50% formamide, 5× SSC (150 m M NaCl, 15 m M trisodium citrate), 50 mM sodium phosphate (pH 7 6), 5× Denhardt's solution, 10% dextran sulphate, and 20 microgram/ml denatured, sheared salmon sperm DNA, followed by washing the filters in O.1× SSC at the same temperature.

These nucleic acids, including nucleotide sequences substantially the same, can be comprised in plasmids, vectors and any other DNA construct which can be used for maintaining, modifying, introducing, or expressing the encoding polypeptide. In particular, vectors wherein said nucleic acid molecule is operatively linked to expression control sequences can allow expression in prokaryotic or eukaryotic host cells of the encoded polypeptide.

The wording “nucleotide sequences substantially the same” includes all other nucleic acid sequences which, by virtue of the degeneracy of the genetic code, also code for the given amino acid sequences. In this sense, the literature provides indications on preferred or optimized codons for recombinant expression (Kane J F et al., 1995).

The nucleic acids and the vectors can be introduced into cells with different purposes, generating transgenic cells and organisms. A process for producing cells capable of expressing a polypeptide of the invention comprises genetically engineering cells with such vectors and nucleic acids.

In particular, host cells (e.g. bacterial cells) can be modified by transformation for allowing the transient or stable expression of the polypeptides encoded by the nucleic acids and the vectors of the invention. Alternatively, said molecules can be used to generate transgenic animal cells or non-human animals (by non-/homologous recombination or by any other method allowing their stable integration and maintenance), having enhanced or reduced expression levels of the polypeptides of the invention, when the level is compared with the normal expression levels. Such precise modifications can be obtained by making use of the nucleic acids of the inventions and of technologies associated, for example, to gene therapy (Meth. Enzymol., vol. 346, 2002) or to site-specific recombinases (Kolb A F, 2002). Model systems based on the Preadipocyte factor-1-like polypeptides disclosed in the present patent application for the systematic study of their function can be also generated by gene targeting into human cell lines (Bunz F, 2002).

Gene silencing approaches may also be undertaken to down-regulate endogenous expression of a gene encoding a polypeptide of the invention. RNA interference (RNA) (Elbashir, S M et al., Nature 2001, 411, 494-498) is one method of sequence specific post-transcriptional gene silencing that may be employed. Short dsRNA oligonucleotides are synthesised in vitro and introduced into a cell. The sequence specific binding of these dsRNA oligonucleotides triggers the degradation of target mRNA, reducing or ablating target protein expression.

Efficacy of the gene silencing approaches assessed above may be assessed through the measurement of polypeptide expression (for example, by Western blotting), and at the RNA level using TaqMan-based methodologies.

The polypeptides of the invention can be prepared by any method known in the art, including recombinant DNA-related technologies, and chemical synthesis technologies. In particular, a method for making a polypeptide of the invention may comprise culturing a host or transgenic cell as described above under conditions in which the nucleic acid or vector is expressed, and recovering the polypeptide encoded by said nucleic acid or vector from the culture. For example, when the vector expresses the polypeptide as a fusion protein with an extracellular or signal-peptide containing proteins, the recombinant product can be secreted in the extracellular space, and can be more easily collected and purified from cultured cells in view of further processing or, alternatively, the cells can be directly used or administered.

The DNA sequence coding for the proteins of the invention can be inserted and ligated into a suitable episomal or non-/homologously integrating vectors, which can be introduced in the appropriate host cells by any suitable means (transformation, transfection, conjugation, protoplast fusion, electroporation, calcium phosphate-precipitation, direct microinjection, etc.). Factors of importance in selecting a particular plasmid or viral vector include: the ease with which recipient cells that contain the vector, may be recognized and selected from those recipient cells which do not contain the vector, the number of copies of the vector which are desired in a particular host; and whether it is desirable to be able to “shuttle” the vector between host cells of different species.

The vectors should allow the expression of the isolated or fusion protein including the polypeptide of the invention in the Prokaryotic or Eukaryotic host cells under the control of transcriptional initiation/termination regulatory sequences, which are chosen to be constitutively active or inducible in said cell. A cell line substantially enriched in such cells can be then isolated to provide a stable cell line.

For Eukaryotic hosts (e.g. yeasts, insect, plant, or mammalian cells), different transcriptional and translational regulatory sequences may be employed, depending on the nature of the host They may be derived form viral sources, such as adenovirus, bovine papilloma virus, Simian virus or the like, where the regulatory signals are associated with a particular gene which has a high level of expression. Examples are the TK promoter of the Herpes virus, the SV40 early promoter, the yeast gal4 gene promoter, etc. Transcriptional initiation regulatory signals may be selected which allow for repression and activation, so that expression of the genes can be modulated. The cells stably transformed by the introduced DNA can be selected by introducing one or more markers allowing the selection of host cells which contain the expression vector. The marker may also provide for phototrophy to an auxotropic host, biocide resistance, e.g. antibiotics, or heavy metals such as copper, or the like. The selectable marker gene can either be directly linked to the DNA gene sequences to be expressed, or introduced into the same cell by co-transfection.

Host cells may be either prokaryotic or eukaryotic. Preferred are eukaryotic hosts, e.g. mammalian cells, such as human, monkey, mouse, and Chinese Hamster Ovary (CHO) cells, because they provide post-translational modifications to proteins, including correct folding and glycosylation. Also yeast cells can carry out post-translational peptide modifications including glycosylation. A number of recombinant DNA strategies exist which utilize strong promoter sequences and high copy number of plasmids which can be utilized for production of the desired proteins in yeast. Yeast recognizes leader sequences in cloned mammalian gene products and secretes peptides bearing leader sequences (i.e., pre-peptides).

The above mentioned embodiments of the invention can be achieved by combining the disclosure provided by the present patent application on the sequence of novel Preadipocyte factor-1-like polypeptides with the knowledge of common molecular biology techniques.

Many books and reviews provides teachings on how to clone and produce recombinant proteins using vectors and Prokaryotic or Eukaryotic host cells, such as some titles in the series “A Practical Approach” published by Oxford University Press (“DNA Cloning 2: Expression Systems”, 1995; “DNA Cloning 4: Mammalian Systems”, 1996; “Protein Expression”, 1999; “Protein Purification Techniques”, 2001).

Moreover, updated and more focused literature provides an overview of the technologies for expressing polypeptides in a high -throughput manner (Chambers S P, 2002; Coleman T A, et al., 1997), of the cell systems and the processes used industrially for the large-scale production of recombinant proteins having therapeutic applications (Andersen D C and Krummen L, 2002, Chu L and Robinson D K, 2001), and of alternative eukaryotic expression systems for expressing the polypeptide of interest, which may have considerable potential for the economic production of the desired protein, such the ones based on transgenic plants (Giddings G, 2001) or the yeast Pichia pastoris (Lin Cereghino G P et al., 2002). Recombinant protein products can be rapidly monitored with various analytical technologies during purification to verify the amount and the quantity of the expressed polypeptides (Baker K N et al., 2002), as well as to check if there is problem of bioequivalence and immunogenicity (Schellekens H, 2002; Gendel S M, 2002).

Totally synthetic Preadipocyte factor-1-like polypeptides are disclosed in the literature and many examples of chemical synthesis technologies, which can be effectively applied for the Preadipocyte factor-1-like polypeptides of the invention given their short length, are available in the literature, as solid phase or liquid phase synthesis technologies, for example, the amino acid corresponding to the carboxy-terminus of the peptide to be synthesized is bound to a support which is insoluble in organic solvents, and by alternate repetition of reactions, one wherein amino acids with their amino groups and side chain functional groups protected with appropriate protective groups are condensed one by one in order from the carboxy-terminus to the amino-terminus, and one where the amino acids bound to the resin or the protective group of the amino groups of the peptides are released, the peptide chain is thus extended in this manner. Solid phase synthesis methods are largely classified by the tBoc method and the Fmoc method, depending on the type of protective group used. Typically used protective groups include tBoc (t-butoxycarbonyl), Cl-Z (2-chlorobenzyloxycarbonyl), Br-Z (2-bromobenzyloxycarbonyl), Bzl (benzyl), Fmoc (9-fluorenylmethoxycarbonyl), Mbh (4,4′-dimethoxydibenzhydryl), Mtr (4-methoxy-2,3,6-trimethylbenzenesulphonyl), Trt (trityl), Tos (tosyl), Z (benzyloxycarbonyl) and Cl2-Bzl (2,6-dichlorobenzyl) for the amino groups; NO2 (nitro) and Pmc (2,2,5,7,8-pentamethylchromane-6-sulphonyl) for the guanidino groups); and tBu (t-butyl) for the hydroxyl groups). After synthesis of the desired peptide, it is subjected to the de-protection reaction and cut out from the solid support. Such peptide cutting reaction may be carried with hydrogen fluoride or tri-fluoromethane sulfonic acid for the Boc method, and with TFA for the Fmoc method.

The purification of the polypeptides of the invention can be carried out by any one of the methods known for this purpose, i.e. any conventional procedure involving extraction, precipitation, chromatography, electrophoresis, or the like. A further purification procedure that may be used in preference for purifying the protein of the invention is affinity chromatography using monoclonal antibodies or affinity groups, which bind the target protein and which are produced and immobilized on a gel matrix contained within a column. Impure preparations containing the proteins are passed through the column. The protein will be bound to the column by heparin or by the specific antibody while the impurities will pass through. After washing, the protein is eluted from the gel by a change in pH or ionic strength. Alternatively, HPLC (High Performance Liquid Chromatography) can be used. The elution can be carried using a water-acetonitrile-based solvent commonly employed for protein purification.

The disclosure of the novel polypeptides of the invention, and the reagents disclosed in connection to them (antibodies, nucleic acids, cells) allows also to screen and characterize compounds that enhance or reduce their expression level into a cell or in an animal.

“Oligonucleotides” refers to either a single stranded polydeoxynucleotide or two complementary polydeoxynucleotide strands which may be chemically synthesized. Such synthetic oligonucleotides have no 5′ phosphate and thus will not ligate to another oligonucleotide without adding a phosphate with an ATP in the presence of a kinase. A synthetic oligonucleotide will ligate to a fragment that has not been dephosphorylated.

The invention includes purified preparations of the compounds of the invention (polypeptides, nucleic acids, cells, etc.). Purified preparations, as used herein, refers to the preparations which contain at least 1%, preferably at least 5%, by dry weight of the compounds of the invention.

The present patent application discloses a series of novel Preadipocyte factor-1-like polypeptides and of related reagents having several possible applications. In particular, whenever an increase in the Preadipocyte factor-1-like activity of a polypeptide of the invention is desirable in the therapy or in the prevention of a disease, reagents such as the disclosed Preadipocyte factor-1-like polypeptides, the corresponding fusion proteins and peptide mimetics, the encoding nucleic acids, the expressing cells, or the compounds enhancing their expression can be used.

Therefore, the present invention discloses pharmaceutical compositions for the treatment or prevention of diseases needing an increase in the Preadipocyte factor-1-like activity of a polypeptide of the invention, which contain one of the disclosed Preadipocyte factor-1-like polypeptides, the corresponding fusion proteins and peptide mimetics, the encoding nucleic acids, the expressing cells, or the compounds enhancing their expression, as active ingredient. The process for the preparation of these pharmaceutical compositions comprises combining the disclosed Preadipocyte factor-1-like polypeptides, the corresponding fusion proteins and peptide mimetics, the encoding nucleic adds, the expressing cells, or the compounds enhancing their expression, together with a pharmaceutically acceptable carrier. Methods for the treatment or prevention of diseases needing an increase in the Preadipocyte factor-1-like activity of a polypeptide of the invention, comprise the administration of a therapeutically effective amount of the disclosed Preadipocyte factor-1-like polypeptides, the corresponding fusion proteins and peptide mimetics, the encoding nucleic acids, the expressing cells, or the compounds enhancing their expression.

Amongst the reagents disclosed in the present patent application, the ligands, the antagonists or the compounds reducing the expression or the activity of polypeptides of the invention have several applications, and in particular they can be used in the therapy or in the diagnosis of a disease associated to the excessive Preadipocyte factor-1-like activity of a polypeptide of the invention.

Therefore, the present invention discloses pharmaceutical compositions for the treatment or prevention of diseases associated to the excessive Preadipocyte factor-1-like activity of a polypeptide of the invention, which contain one of the ligands, antagonists, or compounds reducing the expression or the activity of such polypeptides, as active ingredient. The process for the preparation of these pharmaceutical compositions comprises combining the ligand, the antagonist, or the compound, together with a pharmaceutically acceptable carrier. Methods for the treatment or prevention of diseases associated to the excessive Preadipocyte factor-1-like activity of the polypeptide of the invention, comprise the administration of a therapeutically effective amount of the antagonist, the ligand or of the compound.

The pharmaceutical compositions of the invention may contain, in addition to Preadipocyte factor-1-like polypeptide or to the related reagent, suitable pharmaceutically acceptable carriers, biologically compatible vehicles and additives which are suitable for administration to an animal (for example, physiological saline) and eventually comprising auxiliaries (like excipients, stabilizers, adjuvants, or diluents) which facilitate the processing of the active compound into preparations which can be used pharmaceutically.

The pharmaceutical compositions may be formulated in any acceptable way to meet the needs of the mode of administration. For example, of biomaterials, sugar-macromolecule conjugates, hydrogels, polyethylene glycol and other natural or synthetic polymers can be used for improving the active ingredients in terms of drug delivery efficacy. Technologies and models to validate a specific mode of administration are disclosed in literature (Davis B G and Robinson M A, 2002; Gupta P et al., 2002; Luo B and Prestwich G D, 2001; Cleland J L et al., 2001; Pillai O and Panchagnula R, 2001).

Polymers suitable for these purposes are biocompatible, namely, they are non-toxic to biological systems, and many such polymers are known. Such polymers may be hydrophobic or hydrophilic in nature, biodegradable, non-biodegradable, or a combination thereof. These polymers include natural polymers (such as collagen, gelatin, cellulose, hyaluronic acid), as well as synthetic polymers (such as polyesters, polyorthoesters, polyanhydrides). Examples of hydrophobic non-degradable polymers include polydimethyl siloxanes, polyurethanes, polytetrafluoroethylenes, polyethylenes, polyvinyl chlorides, and polymethyl methaerylates. Examples of hydrophilic non-degradable polymers include poly(2-hydroxyethyl methacrylate), polyvinyl alcohol, poly(N-vinyl pyrrolidone), polyalkylenes, polyacrylamide, and copolymers thereof. Preferred polymers comprise as a sequential repeat unit ethylene oxide, such as polyethylene glycol (PEG).

Any accepted mode of administration can be used and determined by those skilled in the art to establish the desired blood levels of the active ingredients. For example, administration may be by various parenteral routes such as subcutaneous, intravenous, intradermal, intramuscular, intraperitoneal, intranasal, transdermal, oral, or buccal routes. The pharmaceutical compositions of the present invention can also be administered in sustained or controlled release dosage forms, including depot injections, osmotic pumps, and the like, for the prolonged administration of the polypeptide at a predetermined rate, preferably in unit dosage forms suitable for single administration of precise dosages.

Parenteral administration can be by bolus injection or by gradual perfusion over time. Preparations for parenteral administration include sterile aqueous or non-aqueous solutions, suspensions, and emulsions, which may contain auxiliary agents or excipients known in the art, and can be prepared according to routine methods. In addition, suspension of the active compounds as appropriate oily injection suspensions may be administered. Suitable lipophilic solvents or vehicles include fatty oils, for example, sesame oil, or synthetic fatty acid esters, for example, sesame oil, or synthetic fatty acid esters, for example, ethyl oleate or triglycerides. Aqueous injection suspensions that may contain substances increasing the viscosity of the suspension include, for example, sodium carboxymethyl cellulose, sorbitol, and/or dextran. Optionally, the suspension may also contain stabilizers. Pharmaceutical compositions include suitable solutions for administration by injection, and contain from about 0.01 to 99.99 percent, preferably from about 20 to 75 percent of active compound together with the excipient

The wording “therapeutically effective amount” refers to an amount of the active ingredients that is sufficient to affect the course and the severity of the disease, leading to the reduction or remission of such pathology. The effective amount will depend on the route of administration and the condition of the patient.

The wording “pharmaceutically acceptable” is meant to encompass any carrier, which does not interfere with the effectiveness of the biological activity of the active ingredient and that is not toxic to the host to which is administered. For example, for parenteral administration, the above active ingredients may be formulated in unit dosage form for injection in vehicles such as saline, dextrose solution, serum albumin and Ringer's solution. Carriers can be selected also from starch, cellulose, talc, glucose, lactose, sucrose, gelatin, malt, rice, flour, chalk, silica gel, magnesium stearate, sodium stearate, glycerol monostearate, sodium chloride, dried skim milk, glycerol, propylene glycol, water, ethanol, and the various oils, including those of petroleum, animal, vegetable or synthetic origin (peanut oil, soybean oil, mineral oil, sesame oil).

It is understood that the dosage administered will be dependent upon the age, sex, health, and weight of the recipient, kind of concurrent treatment, if any, frequency of treatment, and the nature of the effect desired. The dosage will be tailored to the individual subject, as is understood and determinable by one of skill in the art. The total dose required for each treatment may be administered by multiple doses or in a single dose. The pharmaceutical composition of the present invention may be administered alone or in conjunction with other therapeutics directed to the condition, or directed to other symptoms of the condition. Usually a daily dosage of active ingredient is comprised between 0.01 to 100 milligrams per kilogram of body weight per day. Ordinarily 1 to 40 milligrams per kilogram per day given in divided doses or in sustained release form is effective to obtain the desired results. Second or subsequent administrations can be performed at a dosage, which is the same, less than, or greater than the initial or previous dose administered to the individual.

Apart from methods having a therapeutic or a production purpose, several other methods can make use of the Preadipocyte factor-1-like polypeptides and of the related reagents disclosed in the present patent application.

In a first example, a method is provided for screening candidate compounds effective to treat a disease related to a Preadipocyte factor-1-like polypeptide of the invention, said method comprising:
(a) contacting host cells expressing such polypeptide, transgenic non-human animals, or transgenic animal cells having enhanced or reduced expression levels of the polypeptide, with a candidate compound and
(b) determining the effect of the compound on the animal or on the cell.

In a second example there is provided a method for identifying a candidate compound as an antagonist/inhibitor or agonist/activator of a polypeptide of the invention, the method comprising:
(a) contacting the polypeptide, the compound, and a mammalian cell or a mammalian cell membrane; and
(b) measuring whether the molecule blocks or enhances the Interaction of the polypeptide, or the response that results from such interaction, with the mammalian cell or the mammalian cell membrane.

In a third example, a method for determining the activity and/or the presence of the polypeptide of the invention in a sample, can detect either the polypeptide or the encoding RNA/DNA. Thus, such a method comprises:
(a) providing a protein-containing sample;
(b) contacting said sample with a ligand of the invention; and
(c) determining the presence of said ligand bound to said polypeptide, thereby determining the activity and/or the presence of polypeptide in said sample.

In an alternative, the method comprises:
(a) providing a nucleic acids-containing sample;
(b) contacting said sample with a nucleic acid of the invention; and
(c) determining the hybridization of said nucleic acid with a nucleic acid into the sample, thereby determining the presence of the nucleic acid in the sample.

In this sense, a primer sequence derived from the nucleotide sequence presented in SEQ ID NO:1 or SEQ ID NO:7 can be used as well for determining the presence or the amount of a transcript or of a nucleic acid encoding a polypeptide of invention in a sample by means of Polymerase Chain Reaction amplification.

A further object of the present invention are kits for measuring the activity and/or the presence of Preadipocyte factor-1-like polypeptide of the invention in a sample comprising one or more of the reagents disclosed in the present patent application: a Preadipocyte factor-1-like polypeptide of the invention, an antagonist, ligand or peptide mimetic, an isolated nucleic acid or the vector, a pharmaceutical composition, an expressing cell, or a compound increasing or decreasing the expression levels.

Such kits can be used for in vitro diagnostic or screenings methods, and their actual composition should be adapted to the specific format of the sample (e.g. biological sample tissue from a patient), and the molecular species to be measured. For example, if it is desired to measure the concentration of the Preadipocyte factor-1-like polypeptide, the kit may contain an antibody and the corresponding protein in a purified form to compare the signal obtained in Western blot. Alternatively, if it is desired to measure the concentration of the transcript for the Preadipocyte factor-1-like polypeptide, the kit may contain a specific nucleic acid probe designed on the corresponding ORF sequence, or may be in the form of nucleic acid array containing such probe. The kits can be also in the form of protein-, peptide mimetic-, or cell-based microarrays (Templin M F et al., 2002; Pellois J P et al., 2002; Blagoev B and Pandey A, 2001), allowing high-throughput proteomics studies, by making use of the proteins, peptide mimetics and cells disclosed in the present patent application.
Therapeutic Uses

SCS0009 nucleic acid molecules. polypeptides, and agonists and antagonists thereof can be used to treat, diagnose, ameliorate, or prevent a number of diseases, disorders, or conditions, including those recited herein.

SCS0009 polypeptide agonists and antagonists include those molecules which regulate SCS0009 polypeptide activity and either increase or decrease at least one activity of the mature form of the SCS0009 polypeptide. Agonists or antagonists may be co-factors, such as a protein, peptide, carbohydrate, lipid, or small molecular weight molecule, which interact with SCS0009 polypeptide and thereby regulate its activity.

Potential polypeptide agonists or antagonists include antibodies that react with either soluble or membrane-bound forms of SCS0009 polypeptides that comprise part or all of the extracellular domains of the said proteins. Molecules that regulate SCS0009 polypeptide expression typically include nucleic acids encoding SCS0009 polypeptide that can act as anti-sense regulators of expression.

The present patent application discloses novel Preadipocyte factor-1-like polypeptides and a series of related reagents that may be useful, as active ingredients in pharmaceutical compositions appropriately formulated, in the treatment or prevention of diseases and conditions in which Preadipocyte factor-1-like proteins are implicated, such as obesity, organomegaly, diabetes (including insulin resistance, hyperinsulinemia, hyperglycemia, hypertriglyceridemia), adrenocortical dysfunction, hypertrophy of cardiac and skeletal muscle, lipodystrophy (see OMIM*269700), and immune system disorders including autoimmune diseases and immunodeficiencies (Jensen et al. showed that DLK1 is expressed in the CNS. Neuroreport. 2001 Dec. 21;12(18):3959-63), developmental defects, cancer, autoimmune thyroid diseases and related disorders such as opthalmopathies and other pathological conditions in which Preadipocyte factor-1 protein is implicated.

Moon et al. Show that Pref-1 shows growth retardation, obesity, blepharophimosis, skeletal malformation, and increased serum lipid metabolites (Yang Soo moon et al. Moleculat and Cellular Biology. 2002. Vol.22(15):5585 -5592). As such SCS0009 nucleic acid molecules, polypeptides, agonists and antagonists thereof may be useful in diagnosing or treating growth retardation, obesity, blepharophimosis, skeletal malformation, and decrease serum lipid metabolites.

Murphy et al. indicate that DLK1 might contribute to the discordant phenotypes associated with uniparetnal disomy (UDP) of chromosome 14 (Murphy S K et al. Hum. Mutat. 2003 Jul.;22(1):92-7.). Furthermore, the paper of Sutton et al. suggests that DLK1 might be involved in the onset of mental retardation and maternal uniparental disomy (Am J Med Genet 2002 Sep. 15;112(1):23-7). In addition, Kobayashi et al. point out that DLK1 may contribute to the scoliosis phenotype observed in UDP (Kobayashi S et al. Gene Cells. 2000 Dec;5(12):1029-37). As such SCS0009 nucleic acid molecules, polypeptides, and agonists and antagonists thereof may be useful in diagnosing or treating mental retardation, maternal uniparental disomy or scoliosis.

Van Limpt showed that DLK1 is highly expressed in a subset of neuroblastoma cell lines (Int. J Cancer. 2003 May 20;105(1):61-9; see also van Limpt V et al. Med Pediatr Oncol. 2000 Dec.;35(6):554-8; and Online Mendelian Inheritance in Man™ (OMIM)*176290: http://www.ncbi.nlm.nih.gov/entrez/query.fcqi?db=OMIM). As such SCS0009 nucleic acid molecules, polypeptides, agonists and antagonists thereof may be useful in diagnosing or treating neuroblastoma, pheochromocytoma, lung tumors, neuroendocrine tumors.

Dogget et al. state that DLK1 is essential for normal hematopoieisis and abnormal expression is a proposed marker of myelodysplastic syndrome (Dogget K L et al. J Cell. Bioichem. 2002;86(1):56-66). As such SCS0009 nucleic acid molecules, polypeptides, agonists and antagonists thereof may be useful in diagnosing or treating myelodysplastic syndrome.

The therapeutic applications of the polypeptides of the invention and of the related reagents can be evaluated (in terms or safety, pharmacokinetics and efficacy) by the means of the in vivo I in vitro assays making use of animal cell, tissues and or by the means of in silico/computational approaches (Johnson D E and Wolfgang G H, 2000), known for the validation of Preadipocyte factor-1-like polypeptides and other biological products during drug discovery and preclinical development.

The invention will now be described with reference to the specific embodiments by means of the following Examples, which should not be construed as in any way limiting the present invention. The content of the description comprises all modifications and substitutions which can be practiced by a person skilled in the art in light of the above teachings and, therefore, without extending beyond the meaning and purpose of the claims.

TABLE I

| Amino | | More Preferred |
|---|---|---|
| Acid | Synonymous Groups | Synonymous Groups |
| Ser | Gly, Ala, Ser, Thr, Pro | Thr, Ser |
| Arg | Asn, Lys, Gln, Arg, His | Arg, Lys, His |
| Leu | Phe, Ile, Val, Leu, Met | Ile, Val, Leu, Met |
| Pro | Gly, Ala, Ser, Thr, Pro | Pro |
| Thr | Gly, Ala, Ser, Thr, Pro | Thr, Ser |
| Ala | Gly, Thr, Pro, Ala, Ser | Gly, Ala |
| Val | Met, Phe, lIe, Leu, Val | Met, lIe, Val, Leu |
| Gly | Ala, Thr, Pro, Ser, Gly | Gly, Ala |
| Ile | Phe, Ile, Val, Leu, Met | Ile, Val, Leu, Met |
| Phe | Trp, Phe, Tyr | Tyr, Phe |
| Tyr | Trp, Phe, Tyr | Phe, Tyr |
| Cys | Ser, Thr, Cys | Cys |
| His | Asn, Lys, Gln, Arg, His | Arg, Lys, His |
| Gln | Glu, Asn, Asp, Gln | Asn, Gln |
| Asn | Glu, Asn, Asp, Gln | Asn, Gln |
| Lys | Asn, Lys, Gln, Arg, His | Arg, Lys, His |
| Asp | Glu, Asn, Asp, Gln | Asp, Glu |
| Glu | Glu, Asn, Asp, Gln | Asp, Glu |
| Met | Phe, Ile, Val, Leu, Met | Ile, Val, Leu, Met |
| Trp | Trp, Phe,Tyr | Trp |

TABLE II

| Amino | |
|---|---|
| Acid | Synonymous Groups |
| Ser | D-Ser, Thr, D-Thr, allo-Thr, Met, D-Met, |
| | Met(O), D-Met(O), L-Cys, |
| Arg | D-Arg, Lys, D-Lys, homo-Arg, D-homo-Arg, |
| | Met, Ile, D-.Met, D-Ile, Orn, D-Orn |
| Leu | D-Leu, Val, D-Val, AdaA, AdaG, Leu, D-Leu, |
| | Met, D-Met |
| Pro | D-Pro, L-I-thioazolidine-4-carbixylic acid, |
| | D-or L-1-oxazolidine-4-carboxylic acid |
| Thr | D-Thr, Ser, D-Ser, allo-Thr, Met,D-Met, |
| | Met(O), D-Met(O), Val, D-Val |
| Ala | D-Ala, Gly, Aib, B-Ala, Acp, L-Cys, D-Cys |
| Val | D-Val, Leu, D-Leu, Ile, D-Ile, Met, D-Met, |
| | AdaA, AdaG |
| Gly | Ala, D-Ala, Pro, D-Pro, Aib, .beta.-Ala, Acp |
| Ile | D-Ile, Val, D-Val, AdaA, AdaG, Leu, D-Leu, |
| | Met, D-Met |
| Phe | D-Phe, Tyr, D-Thr, L-Dopa, His, D-His, Trp, |
| | D-Trp, Trans-3,4, or 5-phenylproline, AdaA, |
| | AdaG, cis-3,4, or 5-phenylproline, Bpa, D- |
| | Bpa |
| Tyr | D-Tyr, Phe, D-Phe, L-Dopa, His, D-His |
| Cys | D-Cys, S—Me—Cys, Met, D-Met, Thr, |
| | D-Thr |
| Gln | D-Gln, Asn, D-Asn, Glu, D-Glu, Asp, D-Asp |
| Asn | D-Asn, Asp, D-Asp, Glu, D-Glu, Gln, D-Gln |
| Lys | D-Lys, Arg, D-Arg, homo-Arg, D-homo-Arg, |
| | Met, D-Met, Ile, D-Ile, Orn, D-Orn |
| Asp | D-Asp, D-Asn, Asn, Glu, D-Glu, Gln, D-Gln |
| Glu | D-Glu, D-Asp, Asp, Asn, D-Asn, Gln, D-Gln |
| Met | D-Met, S—Me—Cys, Ile, D-Ile, Leu, |
| | D-Leu, Val, D-Val |

### EXAMPLES

### Example 1

Sequences of EGF protein domains from the ASTRAL database (Brenner S E et al. “The ASTRAL compendium for protein structure and sequence analysis” Nucleic Acids Res. 2000 Jan 1; 28 (1): 254-6) were used to search for homologous protein sequences in genes predicted from human genome sequence (Celera database). The protein sequences were obtained from the gene predictions and translations thereof as generated by one of three programs: the Genescan (Burge C, Karlin S., “Prediction of complete gene structures in human genomic DNA, J Mol Biol. 1997 Apr. 25;268(i):78-94) Grail (Xu Y, Uberbacher E C., <<Automated gene identification in large-scale genomic sequences”, J Comput Biol. 1997 Fall;4(3):325-38) and Fgenesh (Proprietary Celera software).

The sequence profiles of the EGF domains were generated using PIMAII (Profile Induced Multiple Alignment; Boston University software, version II, Das S and Smith T F 2000), an algorithm that aligns homologous sequences and generates a sequence profile. The homology was detected using PIMAII that generates global-local alignments between a query profile and a hit sequence. In this case the algorithm was used with the profile of the EGF functional domain as a query. PIMAII compares the query profile to the database of gene predictions translated into protein sequence and can therefore identify a match to a DNA sequence that contains that domain. Further comparison by BLAST (Basic Local Alignment Search Tool; NCBI version 2) of the sequence with known EFG containing proteins identified the closets homolog (Gish W, States D J. “Identification of protein coding regions by database similarity search.”, Nat Genet. 1993 Mar;3(3):266-72; Pearson W R, Miller W., “Dynamic programming algorithms for biological sequence comparison.”, Methods Enzymol. 1992;210:575 -601; Altschul S F et al., “Basic local alignment search tool”, J Mol Biol. 1990 Oct. 5;215(3):403-10). PIMAII parameters used for the detection were the PIMA prior amino acids probability matrix and a Z-cutoff score of 10. BLAST parameters used were: Comparison matrix=BLOSUM62; word length=3; .E value cutoff=10; Gap opening and extension=default; No filter.

Once the functional domain was identified in the sequence, the genes were re-predicted with the genewise algorithm using the sequence of the closets homolog (Birney E et al., “PairWise and SearchWise: finding the optimal alignment in a simultaneous comparison of a protein profile against all DNA translation frames.”, Nucleic Acids Res. 1996 Jul. 15;24(14):2730-9)

The profiles for homologous EGF domains were generated automatically using the PSI-BLAST (Altshul et al 1997) scripts written in PERL (Practical Extraction and Report Language) and PIMAII.

A total of 55 predicted genes out of the 464 matching the original query generated on the basis of EGF domain profiles were selected since they were judged as potentially novel.

The novelty of the protein sequences was finally assessed by searching protein databases (SwissProt/Trembl, Human IPI and Derwent GENESEQ) using BLAST and a specific annotation has been attributed on the basis of amino acid sequence homology.

### Example 2

One sequence isolated by the methodology set out in Example 1 is that referred to herein as SCS0009 polypeptide sequence.

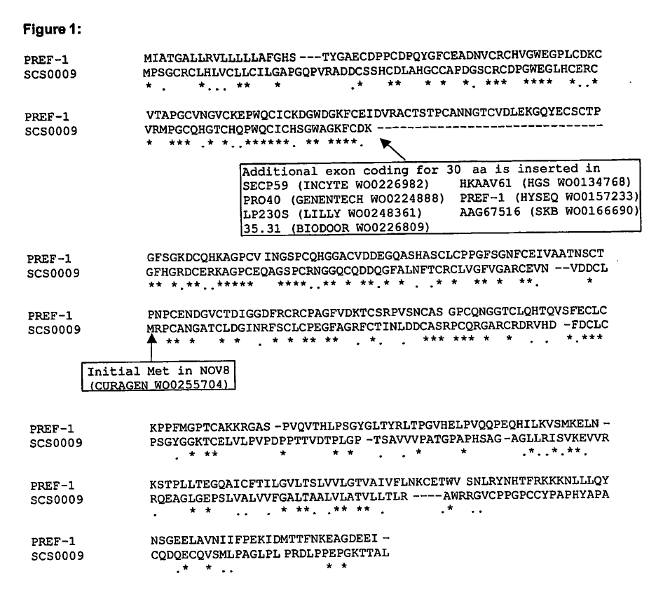

The protein corresponds to a shorter version of longer sequence that is identified in a number of patent applications, essentially lacking the third of the six EGF repeats present in the central region of the protein (see FIG. 1). Some fragments were identified on the basis of the homology with EGF.

Amongst these patent applications, WO0157233 (HYSEQ) describes a protein having a homology with Pref-1 of about 50%, and portions homologous to many different proteins, such as wheat germ agglutinin, laminin, coagulation factors, fibrillin, TNF-RI, IGF-R1, and e-selectin.

Preadipocyte factor-1 (Pref-1, SWISSPROT Q09163; also called Adipocyte differentiation inhibitor protein, Delta-like protein, and fetal antigen 1), is a membrane protein known to inhibit adipocyte differentiation and mediate GH antiadipogenic effects. Knock-out mice present growth retardation, obesity, blepharophimosis, skeletal malformation, and increased serum lipid metabolites, suggesting that Prf-1 is important for homeostasis of adipose tissue mass. Several alternative splicing variants are known, but mostly differ in the C-terminal region, comprising the transmembrane/intracellular sequences.

### Example 3

### Identification and Cloning of SCS0009 and of Splice Variants SCS0009-SV3 and SCS0009-SV4

3.1 Introduction

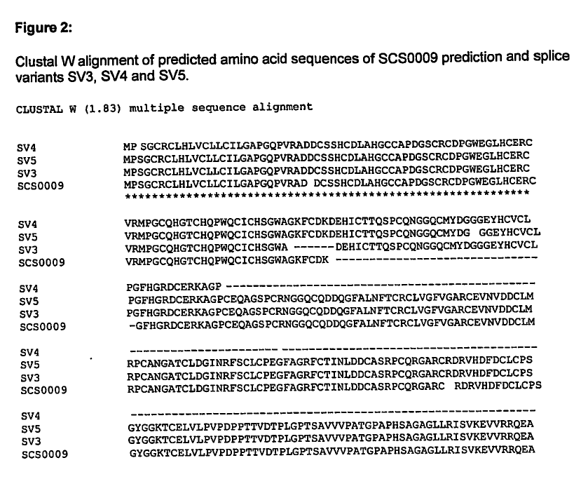

SCS0009 is a 1663 nucleotide cDNA prediction spanning 6 exons, encoding an EGF domain containing protein of 352 amino acids, with homology to preadipocyte factor-1/delta-like protein. SCS0009 is a splice variant of sequences in Celera (hCP1782513.1), NAgeneseq (AAH78208), Mgeneseq (ADA06923) and Swissprot (AAQ88493). The latter sequences contain an additional 31 amino acids occurring between amino acids 90 and 91 of the prediction. The sequence with the 31 amino acid insertion is called SCS0009-SV5. Two image clones were also identified which appear to be splice variants of SCS0009. SCS0009-SV3 (Image clone 5478078) is identical to SV5 except that it contains a 6 amino acid deletion after amino acid 84. SCS0009 -SV4 (Image clone 3349698) is a truncated version of SCS0009-SV5. The alignment of these splice variants is shown in FIG. 2.

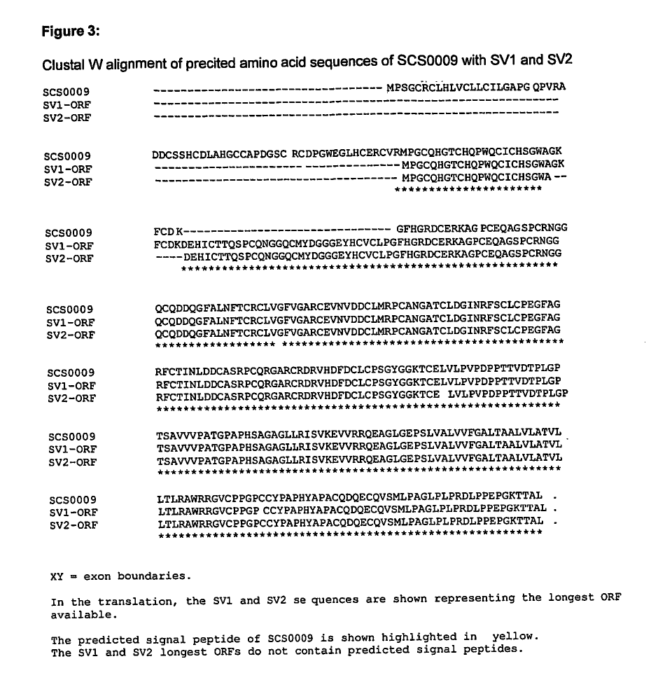

In addition to splice variants 3, 4 and 5 we also identified 2 further splice variants which lacked the first 62 amino acids of the SCS0009 prediction and hence did not contain the predicted signal peptide sequence. SV1 also contained the 31 amino acid insertion seen in the other splice variants. SV2 was identical to SV1 except for the deletion of 6 amino acids upstream of the 31 amino acid insertion. The alignment of SCS0009 with SV1 and SV2 shown in FIG. 3.

Expression constructs for SCS0009 and its splice variants were generated as follows: Image clone 5478078 was used as a template to generate a C-terminal 6HIS tagged version of SCS0009-SV3. Image clone 3349698 was used as a template to generate a C-terminal 6HIS tagged version of SCS0009-SV4. SCS0009-SV5 was engineered from SV3. SCS0009 was engineered from SV5.

3.2 Cloning of SCS0009-SV3

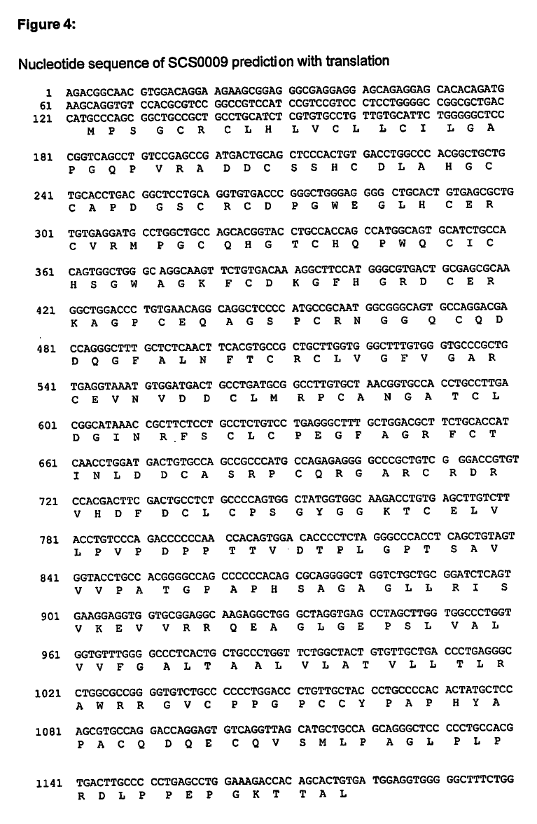

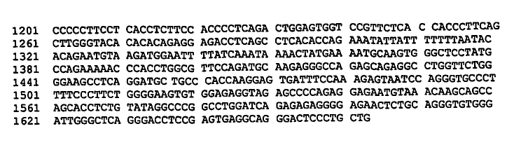

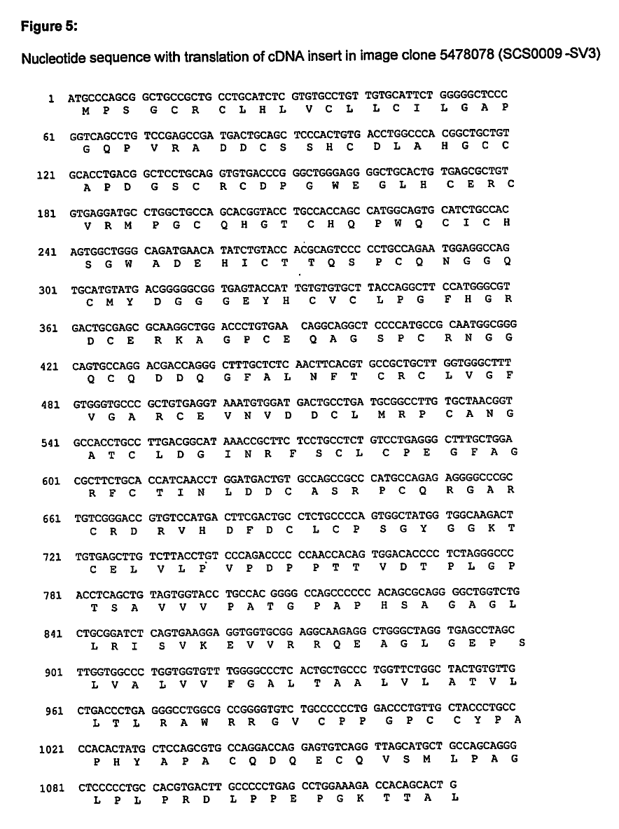

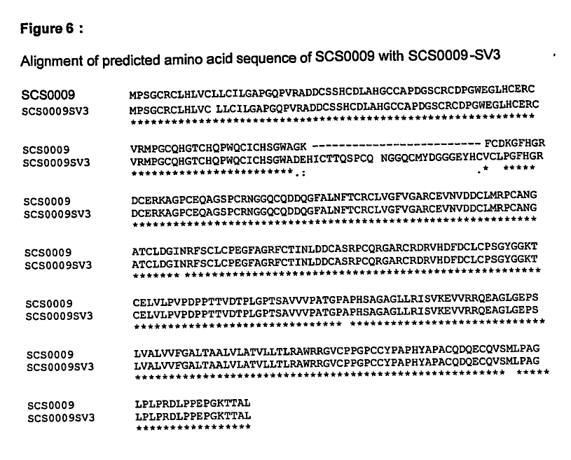

The SCS0009 is a 1663 nucleotide cDNA prediction spanning 6 exons, encoding an EGF domain containing protein of 352 amino acids, with homology to preadipocyte factor-1/delta-like protein. An IMAGE clone (5478078), which contained the SCS0009 coding sequence (FIG. 4), was purchased from ATCC (plasmid ID 14670) and sequence verified using SP6 and T7 primers (table 1). The cDNA insert in the image clone differs from the SCS0009 prediction in that it has a deletion of 18 nucleotides (6 amino acids) at the end of exon 3. It also contains a 5′ extension of 93 nucleotides In exon 4 (resulting in a 31 amino acid insertion) (FIG. 5) and so appears to be a splice variant of SCS0009 which has been called SCS0009 -SV3 (FIG. 6)
3.2.1 Construction of Mammalian Cell Expression Vectors for SCS00009-SV3

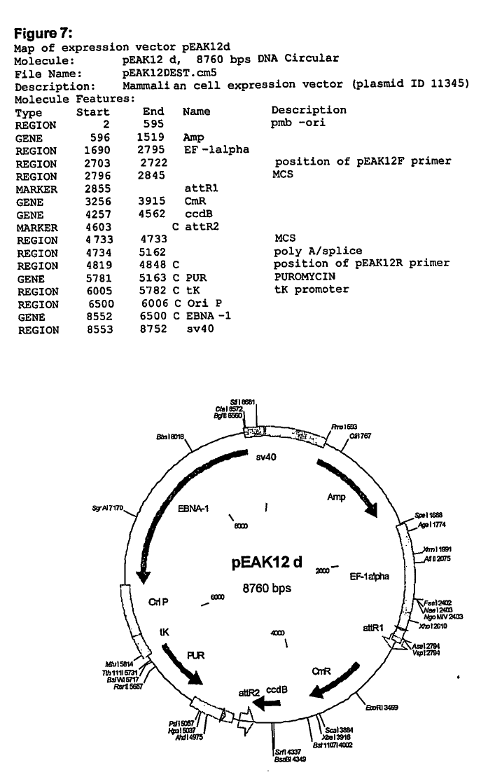

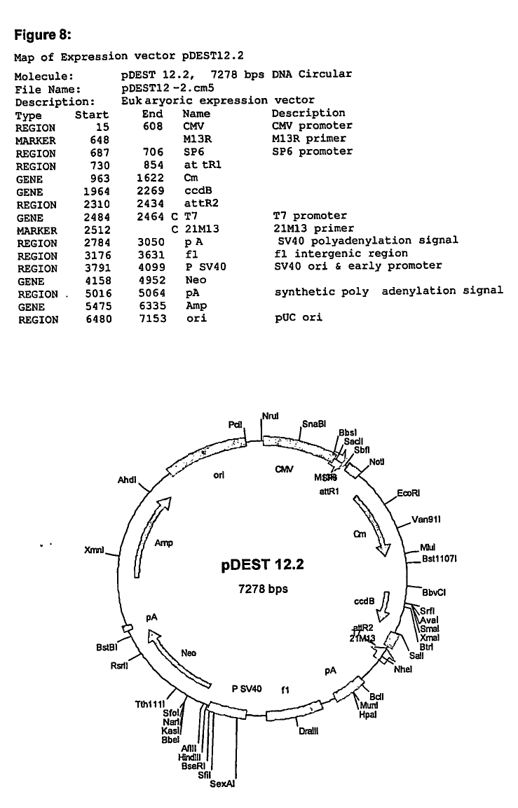

Plasmid 14670 was used as a PCR template to generate pEAK12d (FIG. 7) and pDEST12.2 (FIG. 8) expression clones containing the SCS0009-SV3 ORF sequence with a 3′ sequence encoding a 6HIS tag using the Gateway™ cloning methodology (Invitrogen).
3.2.2 Generation of Gateway Compatible SCS0009-SV3 ORF Fused to an in Frame 6HIS tag Sequence.

The first stage of the Gateway cloning process involves a two step PCR reaction which generates the ORF of SCS0009-SV3 flanked at the 5′ end by an attB1 recombination site and Kozak sequence, and flanked at the 3′ end by a sequence encoding an in frame 6 histidine (6HIS) tag, a stop codon and the attB2 recombination site (Gateway compatible cDNA). The first PCR reaction (in a final volume of 50 μl) contains: 1 μl of plasmid 14670, 1.5 μl dNTPs (10 mM), 10 μl of 10× Pfx polymerase buffer, 1 μl MgSO4 (50 mM), 0.5 μl each of gene specific primer (100 μM) (SCS0009-SV3-EX1 and SCS0009-SV3-EX2), and 0.5 μl Platinum Pfx DNA polymerase (Invitrogen). The PCR reaction was performed using an initial denaturing step of 95° C. for 2 min, followed by 12 cycles of 94° C. for 15 s; 55° C. for 30 s and 68° C. for 2 min; and a holding cycle of 4° C. The amplification products were visualized on 0.8 % agarose gel in 1× TAE buffer (Invitrogen) and a product migrating at the predicted molecular mass was purified from the gel using the Wizard PCR Preps DNA Purification System (Promega) and recovered in 50 μl sterile water according to the manufacturer's instructions.

The second PCR reaction (in a final volume of 50 μl) contained 10 μl purified PCR 1 product, 1.5 μl dNTPs (10 mM), 5 μl of 10× Pfx polymerase buffer, 1 μl MgSO4 (50 mM), 0.5 μl of each Gateway conversion primer (100 μM) (GCP forward and GCP reverse) and 0.5 μl of Platinum Pfx DNA polymerase. The conditions for the 2nd PCR reaction were: 95° C. for 1 min; 4 cycles of 94° C., 15 sec; 50° C., 30 sec and 68° C. for 2 min; 25 cycles of 94° C., 15 sec; 55° C., 30 sec and 68° C., 2 min; followed by a holding cycle of 4° C. PCR products were gel purified using the Wizard PCR prep DNA purification system (Promega) according to the manufacturer's instructions.
3.2.3 Subcloning of Gateway Compatible SCS0009-SV3 ORF into Gateway Entry Vector pDONR221 and Expression Vectors pEAK12d and pDEST12.2

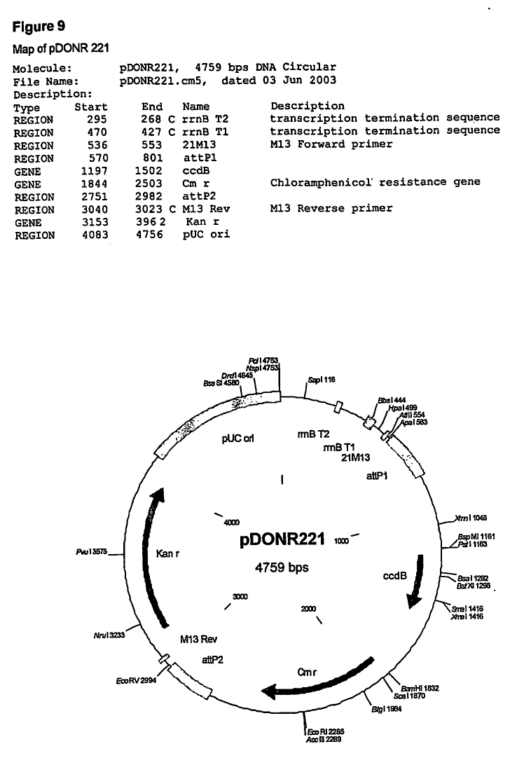

The second stage of the Gateway cloning process involves subcloning of the Gateway modified PCR product into the Gateway entry vector pDONR221 (Invitrogen, FIG. 9) as follows: 5 μl of purified product from PCR2 were incubated with 1.5 μl pDONR221 vector (0.1 μg/μl), 2 μl BP buffer and 1.5 μl of BP clonase enzyme mix (Invitrogen) in a final volume of 10 μl at RT for 1 h. The reaction was stopped by addition of proteinase K 1 μl (2 μg/μl) and incubated at 37° C. for a further 10 min. An aliquot of this reaction (1 μl) was used to transform E. coli DH10B cells by electroporation as follows: a 25 μl aliquot of DH10B electrocompetent cells (Invitrogen) was thawed on ice and 1 μl of the BP reaction mix was added. The mixture was transferred to a chilled 0.1 cm electroporation cuvette and the cells electroporated using a BioRad Gene-Pulser™ according to the manufacturer's recommended protocol. SOC media (0.5 ml), which had been pre-warmed to room temperature, was added immediately after electroporation. The mixture was transferred to a 15 ml snap-cap tube and incubated, with shaking (220 rpm) for 1 h at 37° C. Aliquots of the transformation mixture (10 μl and 50 μl) were then plated on L-broth (LB) plates containing kanamycin (40 μg/ml) and incubated overnight at 37° C.

Plasmid mini-prep DNA was prepared from 5 ml cultures from 6 of the resultant colonies using a Qiaprep Turbo 9600 robotic system (Qiagen). Plasmid DNA (150-200 ng) was subjected to DNA sequencing with 21M13 and M13Rev primers using the BigDyeTerminator system (Applied Biosystems cat. no. 4390246) according to the manufacturer's instructions. The primer sequences are shown in Table 1. Sequencing reactions were purified using Dye -Ex columns (Qiagen) or Montage SEQ 96 cleanup plates (Millipore cat no. LSKS09624) then analyzed on an Applied Biosystems 3700 sequencer.

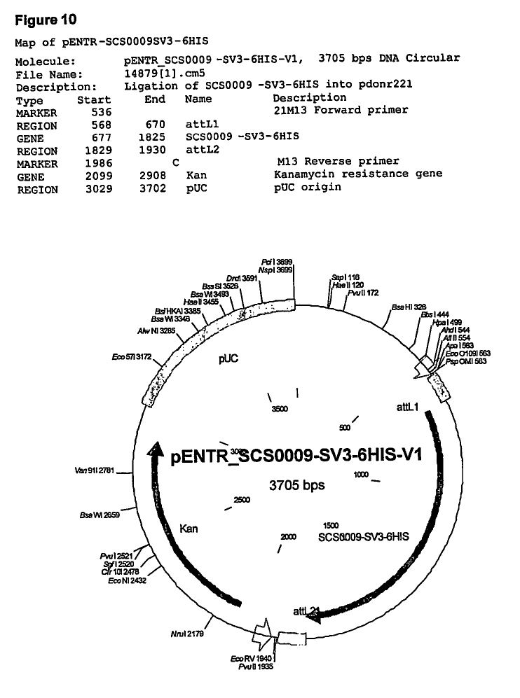

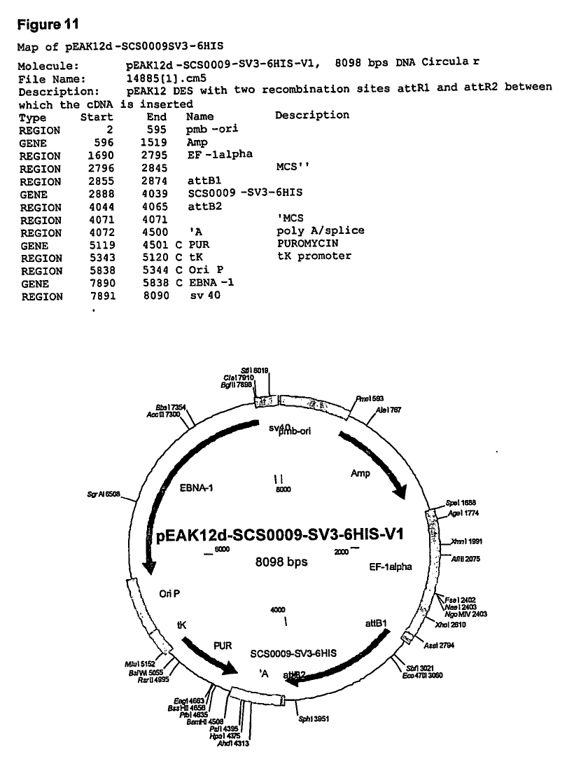

Plasmid eluate (2 μl or approx. 150 ng) from one of the clones which contained the correct sequence (pENTR-SCS0009-SV3-6HIS, plasmid ID 14879, FIG. 10) was then used in a recombination reaction containing 1.5 μl of either pEAK12d vector or pDEST12.2 vector (FIGS. 3 & 4) (0.1 μg/μl), 2 μl LR buffer and 1.5 μl of LR clonase (Invitrogen) in a final volume of 10 μl. The mixture was incubated at RT for 1 h, stopped by addition of proteinase K (2 μg) and incubated at 37° C. for a further 10 min. An aliquot of this reaction (1 ul) was used to transform E. coli DH10B cells by electroporation as follows: a 25 μl aliquot of DH10B electrocompetent cells (Invitrogen) was thawed on ice and 1 μl of the LR reaction mix was added. The mixture was transferred to a chilled 0.1 cm electroporation cuvette and the cells electroporated using a BioRad Gene-Pulser™ according to the manufacturer's recommended protocol. SOC media (0.5 ml), which had been pre-warmed to room temperature, was added immediately after electroporation. The mixture was transferred to a 15 ml snap-cap tube and incubated, with shaking (220 rpm) for 1 h at 37° C. Aliquots of the transformation mixture (10 μl and 50 μl) were then plated on L-broth (LB) plates containing ampicillin (100 μg/ml) and incubated overnight at 37° C.

Plasmid mini-prep DNA was prepared from 5 ml cultures from 6 of the resultant colonies subcloned in each vector using a Qiaprep Turbo 9600 robotic system (Qiagen). Plasmid DNA (200-500 ng) in the pEAK12d vector was subjected to DNA sequencing with pEAK12F, pEAK12R and SCS0009-SV3-SP1 primers as described above. Plasmid DNA (200-500 ng) in the pDEST12.2 vector was subjected to DNA sequencing with 21M13 and M13Rev and SCS0009-SV3-SP1 primers as described above. Primer sequences are shown in Table 1.

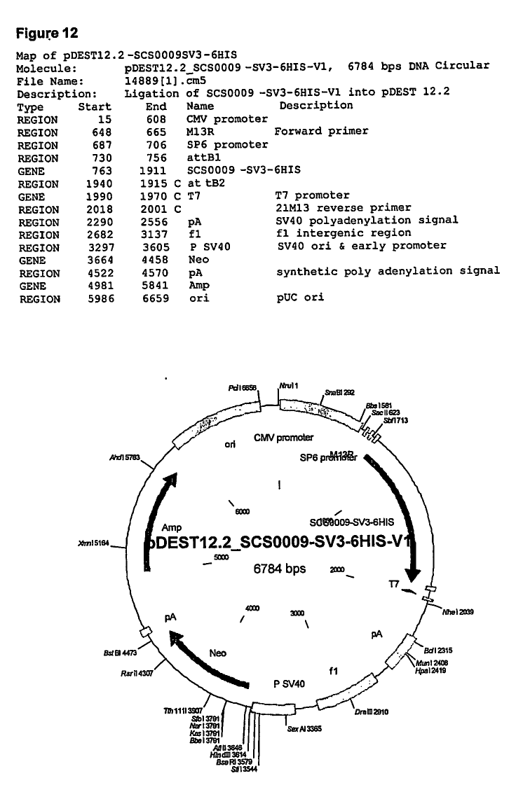

CsCl gradient purified maxi-prep DNA was prepared from a 500 ml culture of one of each of the sequence verified clones (pEAK12d-SCS0009-SV3-6HIS, plasmid ID number 14885, FIG. 11, and pDEST12.2-SCS0009-SV3-6HIS, plasmid ID 14889, FIG. 12) using the method described by Sambrook J. et al., 1989 (in Molecular Cloning, a Laboratory Manual, 2^{nd} edition, Cold Spring Harbor Laboratory Press), Plasmid DNA was resuspended at a concentration of 1 μg/μl in sterile water (or 10 mM Tris-HCl pH 8.5) and stored at '20° C.

TABLE III
SCS0009-SV3 cloning and sequencing primers

| | |
|---|---|
| Primer | Sequence (5′ - 3′) |

| | |
|---|---|
| SCS0009SV3-EX1 | AA GCA GGC TTC GCC ACC ATG CCC AGC |
| | GGC TGC CGC TG |
| SCS0009SV3-EX2 | GTG ATG GTG ATG GTG CAG TGC TGT GGT |
| | CTT TCC AG |
| GCP Forward | G GGG ACA AGT TTG TAC AAA AAA GCA |
| | GGC TTC GCC ACC |
| GCP Reverse | GGG GAC CAC TTT GTA CAA GAA AGC TGG |
| | GTT TCA ATG GTG |
| | ATG GTG ATG GTG |
| SCS0000SV3-SP1 | TGA TGC GGC CTT GTG CTA AC |
| pEAK12F | GCC AGC TTG GCA CTT GAT GT |
| pEAK12R | GAT GGA GGT GGA CGT GTC AG |
| 21M13 | TGT AAA ACG ACG GCC AGT |
| M13REV | CAG GAA ACA GCT ATG ACC |
| T7 primer | TAA TAC GAC TCA CTA TAG GG |
| SP6 primer | ATT TAG GTG ACA CTA TAG |

Underlined sequence = Kozak sequence
Bold = Stop codon
Italic sequence = His tag

3.3 Cloning of SCS0009-SV4

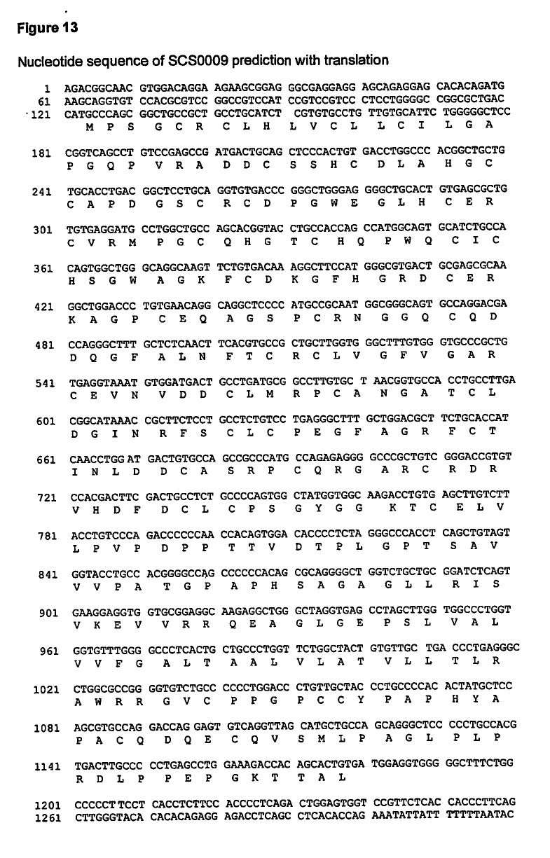

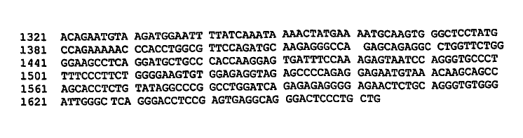

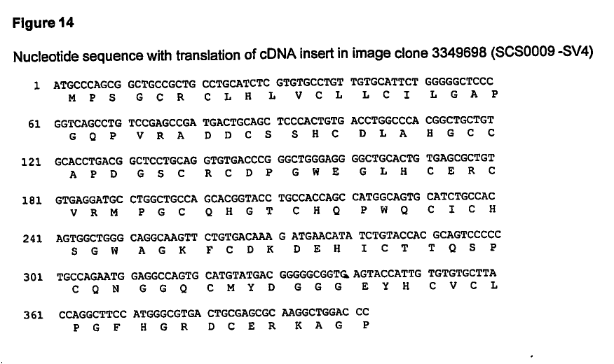

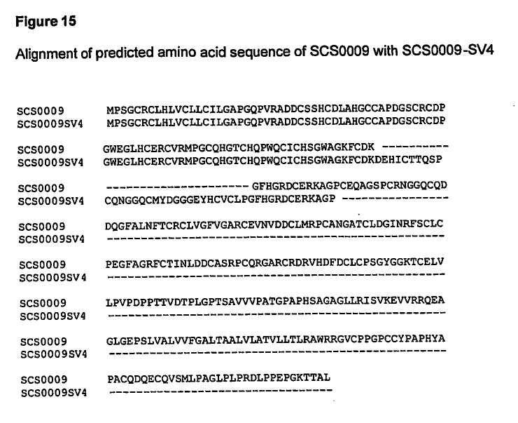

The SCS0009 is a 1663 nucleotide cDNA prediction spanning 6 exons, encoding an EGF domain containing protein of 352 amino acids, with homology to preadipocyte factor-1/delta-like protein. An IMAGE clone (3349698), which contained the SCS0009 coding sequence (FIG. 13), was purchased from ATCC (plasmid ID 14680) and sequence verified using SP6 and T7 primers (table 1). The cDNA insert in the image clone differs from the SCS0009 prediction in that it contains a 5′ extension of 93 nucleotides in exon 4 of the prediction (resulting in a 31 amino acid insertion). There is also a deletion of 133 bp near to the end of exon 4 which leads to a frameshift and immediate stop codon producing a truncated version of SCS0009 (FIG. 14). The cDNA of the image clone therefore appears to encode a splice variant of SCS0009 which we have called SCS0009-SV4. The alignment of SCS0009 with the splice variant is shown in FIG. 15.
3.3.1 Construction of Mammalian Cell Expression Vectors for SCS00009-SV4

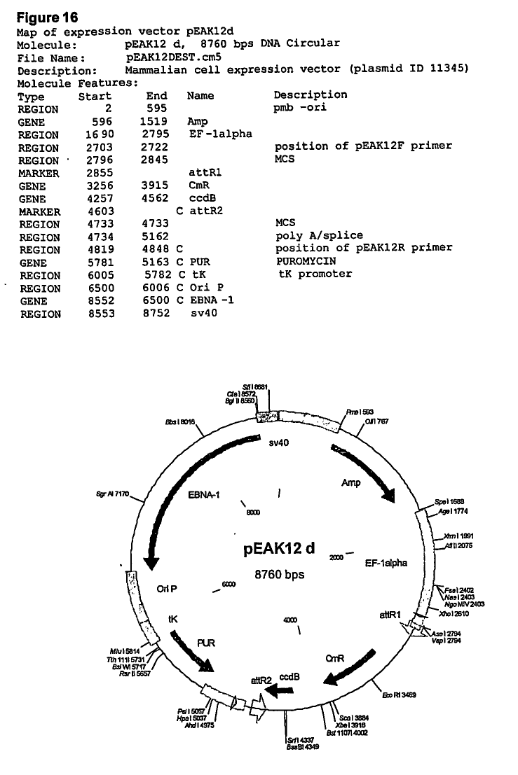

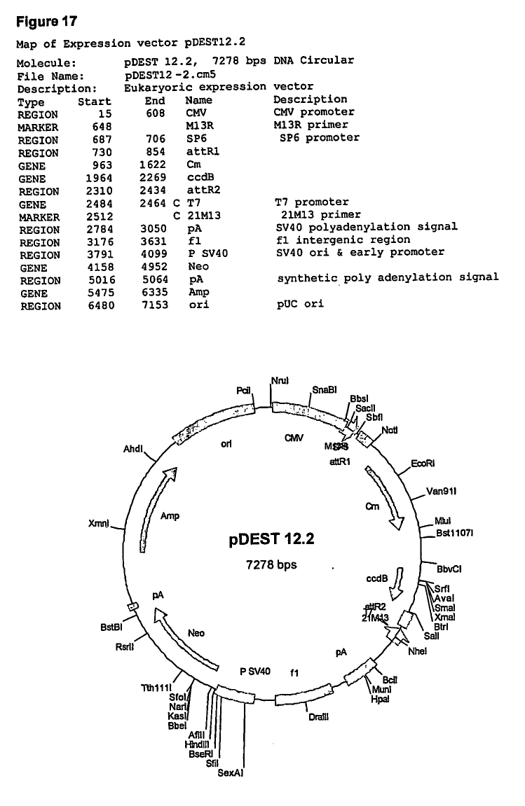

Plasmid 14680 was used as a PCR template to generate pEAK12d (FIG. 16) and pDEST12.2 (FIG. 17) expression clones containing the SCS0009 -SV4 ORF sequence with a 3′ sequence encoding a 6HIS tag using the Gateway™ cloning methodology (Invitrogen).
3.3.2 Generation of Gateway Compatible SCS0009-SV4 ORF Fused to an in Frame 6HIS tag sequence

The first stage of the Gateway cloning process involves a two step PCR reaction which generates the ORF of SCS0009-SV4 flanked at the 5′ end by an attB1 recombination site and Kozak sequence, and flanked at the 3′ end by a sequence encoding an in frame 6 histidine (6HIS) tag, a stop codon and the attB2 recombination site (Gateway compatible cDNA). The first PCR reaction (in a final volume of 50 μl) contains: 1 μl of plasmid 14680, 1.5 μl dNTPs (10 mM), 10 μl of 10× Pfx polymerase buffer, 1 μl MgSO4 (50 mM), 0.5 μl each of gene specific primer (100 μM) (SCS0009-SV4-EX1 and SCS0009-SV4-EX2), and 0.5 μl Platinum Pfx DNA polymerase (Invitrogen). The PCR reaction was performed using an initial denaturing step of 95° C. for 2 min, followed by 12 cycles of 94° C. for 15 s; 55° C. for 30 s and 68° C. for 2 min; and a holding cycle of 4° C. The amplification products were visualized on 0.8% agarose gel in 1 × TAE buffer (Invitrogen) and a product migrating at the predicted molecular mass was purified from the gel using the Wizard PCR Preps DNA Purification System (Promega) and recovered in 50 μl sterile water according to the manufacturer's instructions.

The second PCR reaction (in a final volume of 50 μl) contained 10 μl purified PCR 1 product, 1.5 μl dNTPs (10 mM), 5 μl of 10× Pfx polymerase buffer, 1 μl MgSO4 (50 mM), 0.5 μl of each Gateway conversion primer (100 μM) (GCP forward and GCP reverse) and 0.5 μl of Platinum Pfx DNA polymerase. The conditions for the 2nd PCR reaction were: 95° C. for 1 min; 4 cycles of 94° C., 15 sec; 50° C., 30 sec and 68° C. for 2 min; 25 cycles of 94° C., 15 sec; 55° C., 30 sec and 68° C., 2 min; followed by a holding cycle of 4° C. PCR products were gel purified using the Wizard PCR prep DNA purification system (Promega) according to the manufacturer's instructions.
3.3.3 Subcloning of Gateway Compatible SCS0009-SV4 ORF into Gateway Entry Vector pDONR221 and Expression Vectors pEAK12d and pDEST12.2

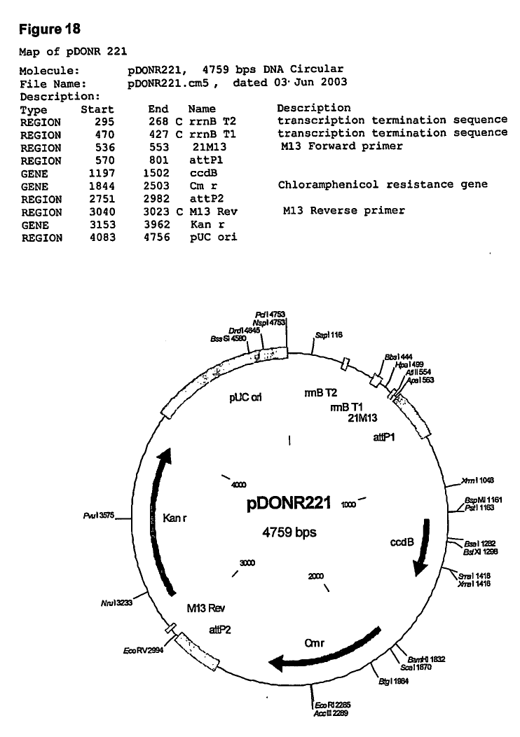

The second stage of the Gateway cloning process involves subcloning of the Gateway modified PCR product into the Gateway entry vector pDONR221 (Invitrogen, FIG. 18) as follows: 5 μl of purified product from PCR2 were incubated with 1.5 μl pDONR221 vector (0.1 μl/μl), 2 μl BP buffer and 1.5 μl of BP clonase enzyme mix (Invitrogen) in a final volume of 10 μl at RT for 1 h. The reaction was stopped by addition of proteinase K 1 μl (2 μg/μl) and incubated at 37° C. for a further 10 min. An aliquot of this reaction (1 μl) was used to transform E. coli DH10B cells by electroporation as follows: a 25 μl aliquot of DH10B electrocompetent cells (Invitrogen) was thawed on ice and 1 μl of the BP reaction mix was added. The mixture was transferred to a chilled 0.1 cm electroporation cuvette and the cells electroporated using a BioRad Gene-Pulser™ according to the manufacturer's recommended protocol. SOC media (0.5 ml), which had been pre-warmed to room temperature, was added immediately after electroporation. The mixture was transferred to a 15 ml snap-cap tube and incubated, with shaking (220 rpm) for 1 h at 37° C. Aliquots of the transformation mixture (10 μl and 50 μl) were then plated on L-broth (LB) plates containing kanamycin (40 μg/ml) and incubated overnight at 37° C.

Plasmid mini-prep DNA was prepared from 5 ml cultures from 6 of the resultant colonies using a Qiaprep Turbo 9600 robotic system (Qiagen). Plasmid DNA (150-200 ng) was subjected to DNA sequencing with 21M13 and M13Rev primers using the BigDyeTerminator system (Applied Biosystems cat. no. 4390246) according to the manufacturer's instructions. The primer sequences are shown in Table 1. Sequencing reactions were purified using Dye-Ex columns (Qiagen) or Montage SEQ 96 cleanup plates (Millipore cat. no. LSKS09624) then analyzed on an Applied Biosystems 3700 sequencer.

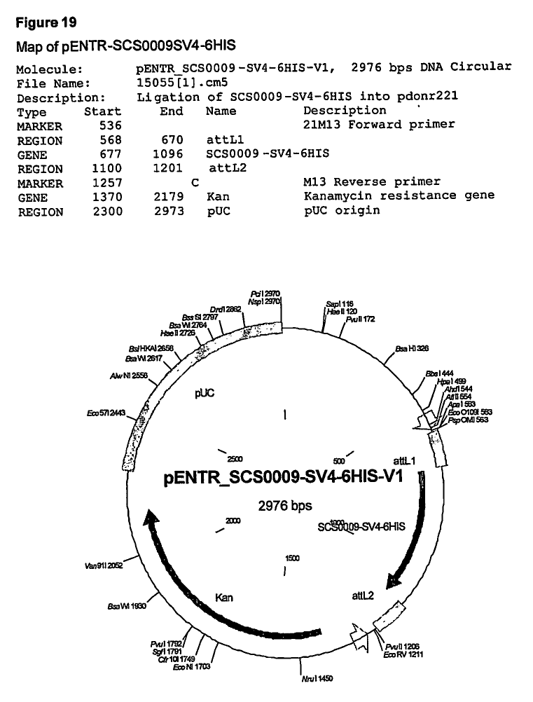

Plasmid eluate (2 μl or approx. 150 ng) from one of the clones which contained the correct sequence (pENTR-SCS0009-SV4-6HIS, plasmid ID 15055, FIG. 19) was then used in a recombination reaction containing 1.5 μl of either pEAK12d vector or pDEST12.2 vector (FIGS. 16 & 17) (0.1 μg/μl), 2 μl LR buffer and 1.5 μl of LR clonase (Invitrogen) in a final volume of 10 μl. The mixture was incubated at RT for 1 h, stopped by addition of proteinase K (2 μg) and incubated at 37° C. for a further 10 min. An aliquot of this reaction (1 μl) was used to transform E. coli DH10B cells by electroporation as follows: a 25 μl aliquot of DH10B electrocompetent cells (Invitrogen) was thawed on ice and 1 μl of the LR reaction mix was added. The mixture was transferred to a chilled 0.1 cm electroporation cuvette and the cells electroporated using a BioRad Gene-Pulser™ according to the manufacturer's recommended protocol. SOC media (0.5 ml), which had been pre-warmed to room temperature, was added immediately after electroporation. The mixture was transferred to a 15 ml snap-cap tube and incubated, with shaking (220 rpm) for 1 h at 37° C. Aliquots of the transformation mixture (10 μl and 50 μl) were then plated on L-broth (LB) plates containing ampicillin (100 μg/ml) and incubated overnight at 37° C.

Plasmid mini-prep DNA was prepared from 5 ml cultures from 6 of the resultant colonies subcloned in each vector using a Qiaprep Turbo 9600 robotic system (Qiagen). Plasmid DNA (200-500 ng) in the pEAK12d vector was subjected to DNA sequencing with pEAK12F and pEAK12R primers as described above. Plasmid DNA (200-500 ng) in the pDEST12.2 vector was subjected to DNA sequencing with 21M13 and M13Rev primers as described above. Primer sequences are shown in Table 1.

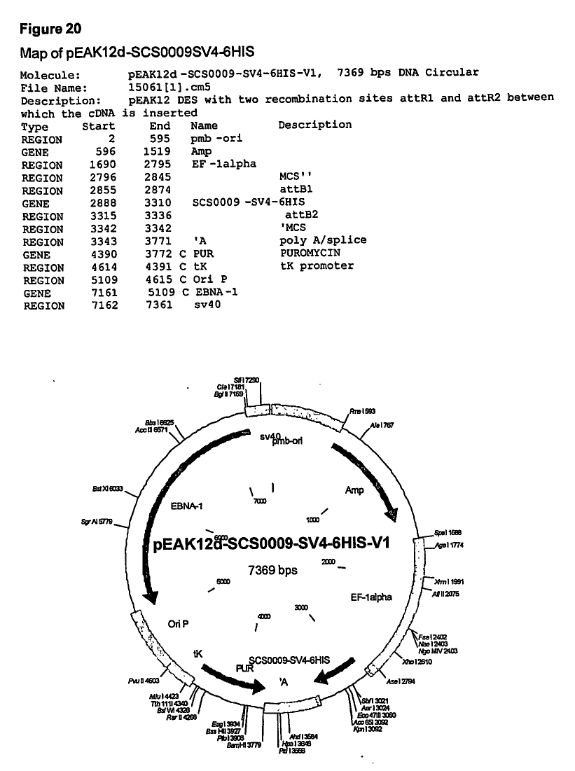

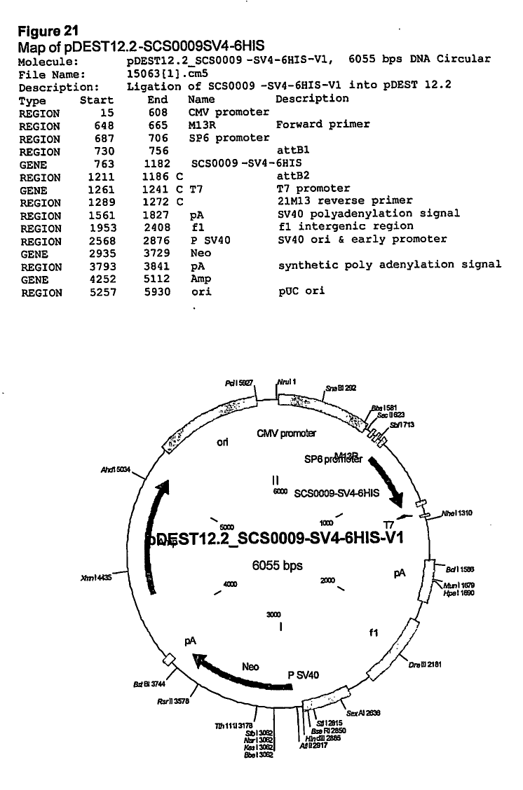

CsCl gradient purified maxi-prep DNA was prepared from a 500 ml culture of one of each of the sequence verified clones (pEAK12d-SCS0009-SV4-6HIS, plasmid ID number 15061, FIG. 20, and pDEST12.2-SCS0009-SV4-6HIS, plasmid ID 15063, FIG. 21) using the method described by Sambrook J. et al., 1989 (in Molecular Cloning, a Laboratory Manual, 2^{nd} edition, Cold Spring Harbor Laboratory Press), Plasmid DNA was resuspended at a concentration of 1 μg/μl in sterile water (or 10 mM Tris-HCl pH 8.5) and stored at −20° C.

TABLE IV
SCS0009-SV4 cloning and sequencing primers

| | |
|---|---|
| Primer | Sequence (5′ - 3′) |

| | |
|---|---|
| SCS0009SV4-EX1 | AA GCA GGC TTC GCC ACC ATG CCC AGC |
| | GGC TGC CGC TG |
| SCS0009SV4-EX2 | GTG ATG GTG ATG GTG GGG TCC AGC CTT |
| | GCG CTC GC |
| GCP Forward | G GGG ACA AGT TTG TAC AAA AAA GCA |
| | GGC TTC GCC ACC |
| GCP Reverse | GGG GAC CAC TTT GTA CAA GAA AGC TGG |
| | GTT TCA ATG GTG |
| | ATG GTG ATG GTG |
| pEAK12F | GCC AGC TTG GCA CTT GAT GT |
| pEAK12R | GAT GGA GGT GGA CGT GTC AG |
| 21M13 | TGT AAA ACG ACG GCC AGT |
| M13REV | CAG GAA ACA GCT ATG ACC |
| T7 primer | TAA TAC GAC TCA CTA TAG GG |
| SP6 primer | ATT TAG GTG ACA CTA TAG |

Underlined sequence = Kozak sequence
Bold = Stop codon
Italic sequence = His tag

3.4 Cloning of SCS0009 by Exon Assembly

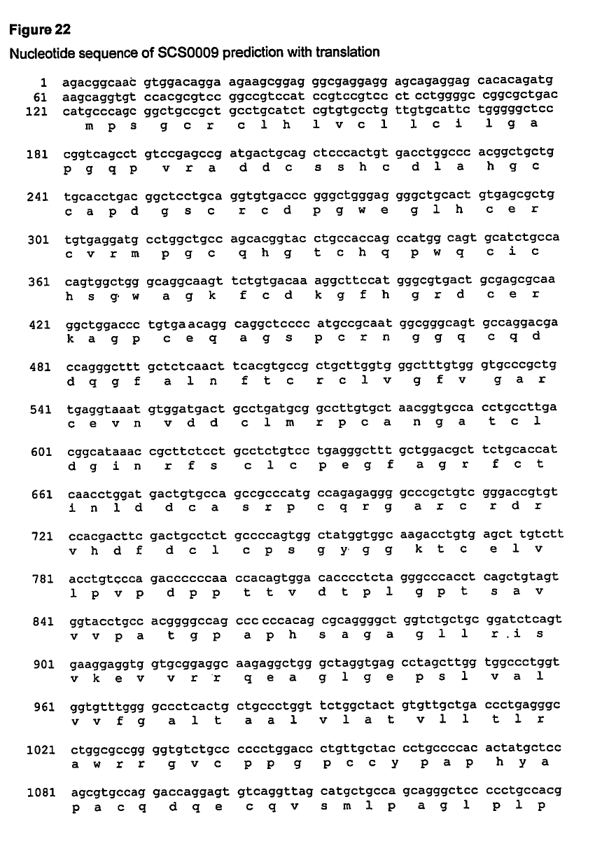

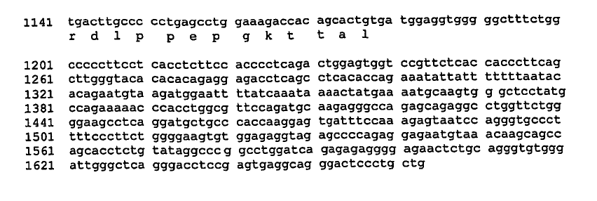

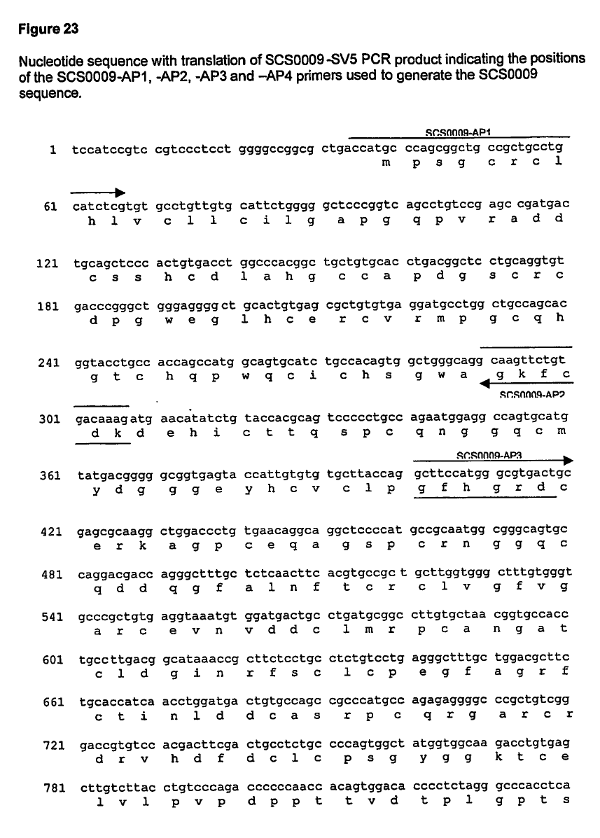

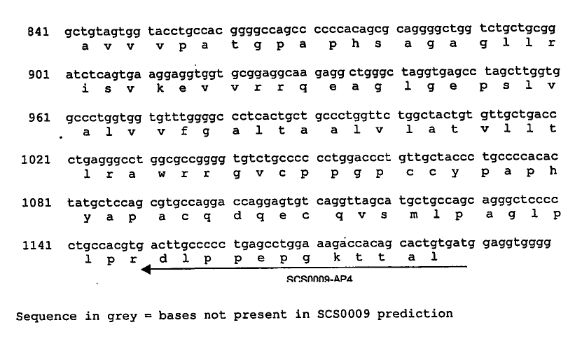

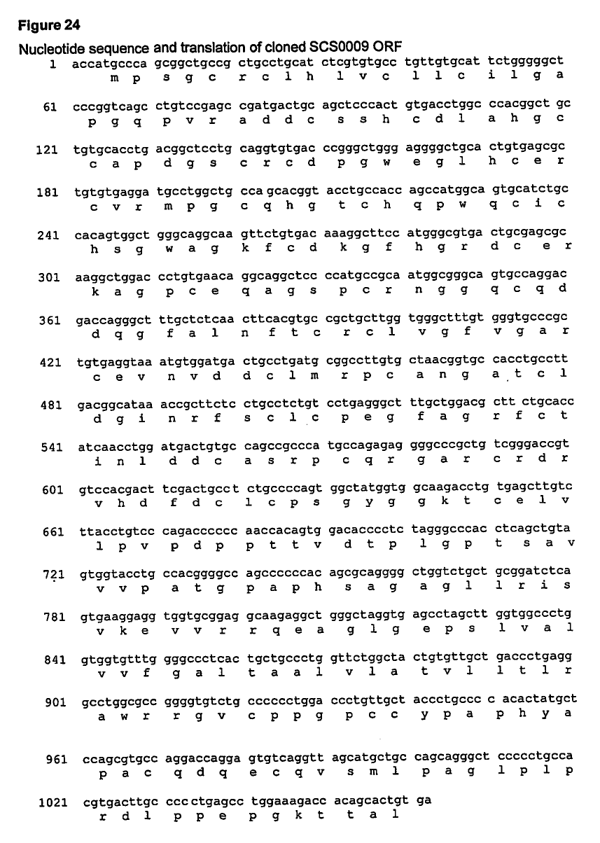

SCS0009 is a prediction of 1663 nucleotides spanning 6 exons which encodes a protein of 352 amino acids with homology to preadipocyte factor-1/delta-like protein (FIG. 22). Various splice variants of the SCS0009 prediction have already been cloned. The version with closest sequence identity to the SCS0009 prediction, called SCS0009-SV5 (plasmid ID. 14846) differs from SCS0009 only by the presence of a 31 amino acid insertion between exons 3 and 4. In order to generate SCS0009 protein:
Exons 1-3 and exons 4-5 of SCS0009 were amplified from plasmid ID. 14846 (pCR4-TOPO-SCS0009-SV5) by PCR (FIG. 23).
The gel-purified exons were mixed and a new PCR reaction was performed to amplify the re-assembled DNA.
The full length PCR product corresponding to the SCS0009 coding sequence (FIG. 24) was subcloned into pCR-BluntII-TOPO cloning vector (Invitrogen) and then sequentially into pDONR 201 (Gateway entry vector) and expression vectors pEAK12d and pDEST12.2. (expression vectors) using the Invitrogen Gateway™ methodology.
3.4.1 PCR Amplification of Exons Encoding SCS0009 from Plasmid ID. 14846 (pCR4-TOPO-SCS0009-SV5)

PCR primers were designed to amplify exons 1-3 and exons 4-5 of SCS0009 (Table 1). The reverse primer for exon 3 (SCS0009-AP2) has an overlap of 18 bp with exon 4 of SCS0009 at its 5′ end. The forward primer for exon 4 (SCS0009 -AP3) has an 18 bp overlap with exon 3 of SCS0009 at its 5′ end.

To generate the product representing exons 1-3 of SCS0009, the PCR reaction was performed in a final volume of 50 μl containing 100 ng of plasmid ID 14846, 1.5 μl of 10 mM dNTPs (Amersham Pharmacia Biotech), 1 μl of MgSO₄ (Invitrogen), 1.5 μl of SCS0009-API (10 μM), 1.5 μl of SCS0009-AP2 (10 μM), 10 μl of 10× Pfx buffer and 0.5 μl of Pfx polymerase (2.5 U/μl) (Invitrogen). The PCR conditions were 94° C. for 5 min; 25 cycles of 94° C. for 15 s, 68° C. for 1 min ; an additional elongation cycle of 68° C. for 7 min; and a holding cycle of 4° C. Reaction products were loaded onto a 0.8% agarose gel (1× TAE) and PCR products of the correct size (292 bp) were gel-purified using the Wizard PCR Preps DNA Purification System (Promega) and recovered in 50 μl sterile water according to the manufacturer's instructions. The product representing exons 4-5 of SCS0009 was produced and purified using the same method except PCR primers SCS0009-AP3/SCS0009-AP4 were used. The SCS0009-AP3/SCS0009-AP4 PCR product was 806 bp.
3.4.2 Assembly of Exons 1-3 and Exons 4-5 to generate the SCS0009 ORF

Exons 1-3 and 4-5 were assembled in a 50 μl PCR reaction containing 2 μl of gel purified exon 1-3 product, 2 μl of gel purified exon 4-5 product, 1.5 μl of 10 mM dNTPs (Amersham Pharmacia Biotech), 1 μl of MgSO₄ (Invitrogen), 1.5 μl of SCS0009-AP1 (10 μM), 1.5 μl of SCS0009-AP4 (10 μM), 10 μl of 10× Pfx buffer and 0.5 μl of Pfx polymerase (2.5 U/μl) (Invitrogen). The PCR conditions were 94° C. for 5 min; 25 cycles of 94° C. for 15 s, 68° C. for 1 min 30 sec; an additional elongation cycle of 68 ° C. for 7 min; and a holding cycle of 4° C. Reaction products were loaded onto a 0.8% agarose gel (1× TAE) and PCR products of the correct size (1062 bp) were gel-purified using the Wizard PCR Preps DNA Purification System (Promega), recovered in 30 μl sterile water according to the manufacturers instructions and stored at −20° C. until subcloning.
3.4.3 Subcloning of PCR Products

The PCR product was subcloned into the topoisomerase I modified cloning vector (pCR-BluntII-TOPO) purchased from the Invitrogen Corporation using the conditions specified by the manufacturer. Briefly, 4 μl of gel purified PCR product was incubated for 15 min at room temperature with 1 μl of TOPO vector and 1 μl salt solution. The reaction mixture was then transformed into E. coli strain TOP10 (Invitrogen) as follows: a 50 μl aliquot of One Shot TOP10 cells was thawed on ice and 2 μl of TOPO reaction was added. The mixture was incubated for 15 min on ice and then heat shocked by incubation at 42° C. for exactly 30 s. Samples were returned to ice and 250 μl of warm (room temperature) SOC media was added. Samples were incubated with shaking (220 rpm) for 1 h at 37° C. The transformation mixture was then plated on L-broth (LB) plates containing kanamycin (40 μg/ml) and incubated overnight at 37° C.
3.4.4 Colony PCR

Colonies were inoculated into 50 μl sterile water using a sterile toothpick. A 10 μl aliquot of the inoculum was then subjected to PCR in a total reaction volume of 20 μl containing 1× AmpliTaq™ buffer, 200 μM dNTPs, 20 pmoles T7 primer, 20 pmoles of SP6 primer, 1 unit of AmpliTaq™ (Perkin Elmer) using an MJ Research DNA Engine. The cycling conditions were as follows: 94° C., 2 min; 30 cycles of 94° C., 30 sec, 48° C., 30 sec and 72° C. for 1 min 30 sec. Samples were maintained at 4° C. (holding cycle) before further analysis. PCR reaction products were analyzed on 1% agarose gels in 1× TAE buffer. Colonies which gave the expected PCR product size (1062 bp cDNA+186 bp due to the multiple cloning site or MCS) were grown up overnight at 37° C. in 5 ml L-Broth (LB) containing kanamycin (40 μg/ml), with shaking at 220 rpm.
3.4.5 Plasmid DNA Preparation and Sequencing

Miniprep plasmid DNA was prepared from 1.5 ml cultures using the FastPlasmid™ Mini Kit (Eppendorf) according to the manufacturer's instructions. Plasmid DNA was eluted in 50 μl of elution buffer (10 mM Tris-Cl, 0.1 mM EDTA, pH 8.5). The DNA concentration was measured using an Spectromax 190 photometer (Molecular Devices). Plasmid DNA (200-500 ng) was subjected to DNA sequencing with the T7 and SP6 primers using the BigDyeTerminator system (Applied Biosystems cat. no. 4390246) according to the manufacturer's instructions. The primer sequences are shown in Table 1. Sequencing reactions were purified using Dye-Ex columns (Qiagen) or Montage SEQ 96 cleanup plates (Millipore cat. no. LSKS09624) then analyzed on an Applied Biosystems 3700 sequencer.

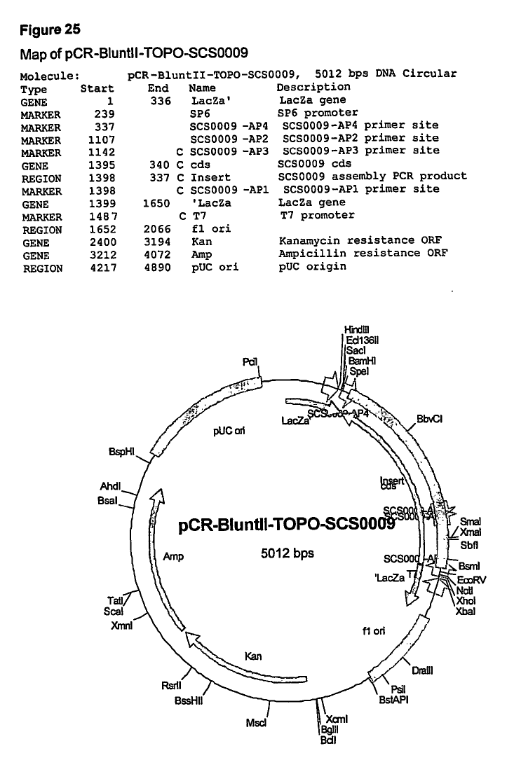

Sequence analysis identified a clone containing 100% match to the predicted SCS0009 sequence. The sequence of the cloned cDNA fragment is shown in FIG. 24. The plasmid map of the cloned PCR product (pCR-BluntII-TOPO-SCS0009) (plasmid ID.14893) is shown in FIG. 25.
3.4.6 Construction of Mammalian Cell Expression Vectors for SCS00009

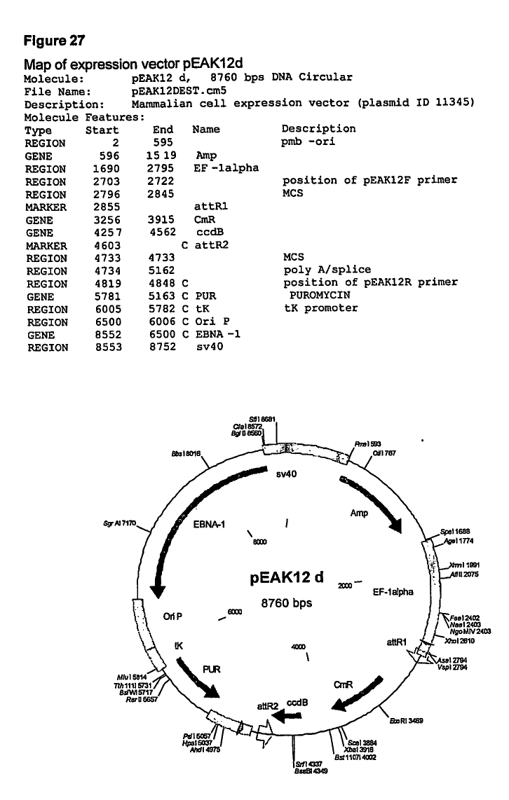

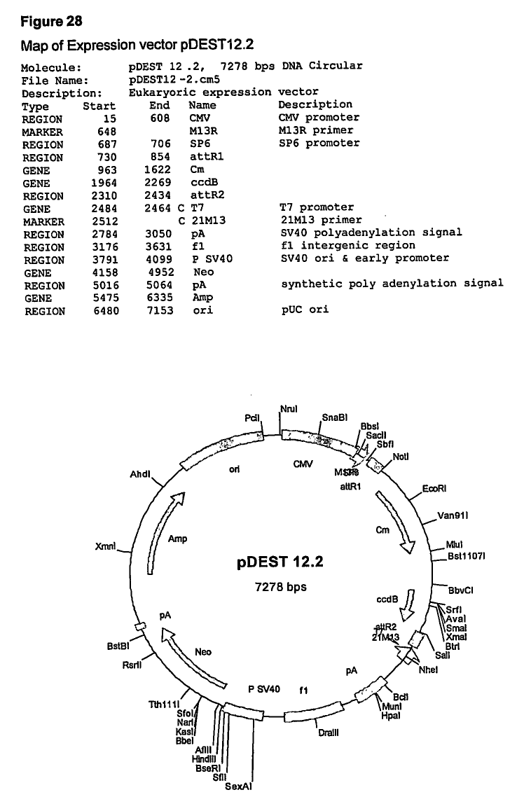

Plasmid 14893 was used as a PCR template to generate pEAK12d (FIG. 27) and pDEST12.2 (FIG. 28) expression clones containing the SCS0009 ORF sequence with a 3′ sequence encoding a 6HIS tag using the Gateway™ cloning methodology (Invitrogen).
3.4.7 Generation of Gateway Compatible SCS0009 ORF Fused to an in Frame 6HIS Tag Sequence

The first stage of the Gateway cloning process involves a two step PCR reaction which generates the ORF of SCS0009 flanked at the 5′ end by an attB1 recombination site and Kozak sequence, and flanked at the 3′ end by a sequence encoding an in-frame 6 histidine (6HIS) tag, a stop codon and the attB2 recombination site (Gateway compatible cDNA). The first PCR reaction (in a final volume of 50 μl ) contains: 1 μl (40 ng) of plasmid 14893, 1.5 μl dNTPs (10 mM), 10 μl of 10× Pfx polymerase buffer, 1 μl MgSO4 (50 mM), 0.5 μl each of gene specific primer (100 μM) (SCS0009-EX1 and SCS0009-EX2), and 0.5 μl Platinum Pfx DNA polymerase (Invitrogen). The PCR reaction was performed using an initial denaturing step of 95° C. for 2 min, followed by 12 cycles of 94° C. for 15 s; 55° C. for 30 s and 68° C. for 2 min; and a holding cycle of 4° C. The amplification products were visualized on 0.8% agarose gel in 1× TAE buffer (Invitrogen) and a product migrating at the predicted molecular mass was purified from the gel using the Wizard PCR Preps DNA Purification System (Promega) and recovered in 50 μl sterile water according to the manufacturer's instructions.

The second PCR reaction (in a final volume of 50 μl ) contained 10 μl purified PCR 1 product, 1.5 μl dNTPs (10 mM), 5 μl of 10× Pfx polymerase buffer, 1 μl MgSO4 (50 mM), 0.5 μl of each Gateway conversion primer (100 μM) (GCP forward and GCP reverse) and 0.5 μl of Platinum Pfx DNA polymerase. The conditions for the 2nd PCR reaction were: 95° C. for 1 min; 4 cycles of 94° C., 15 sec; 50° C., 30 sec and 68° C. for 2 min; 25 cycles of 94° C., 15 sec; 55° C. , 30 sec and 68° C., 2 min; followed by a holding cycle of 4° C. PCR products were gel purified using the Wizard PCR prep DNA purification system (Promega) according to the manufacturer's instructions.
3.4.8 Subcloning of Gateway Compatible SCS0009 ORF into Gateway Entry Vector pDONR221 and Expression Vectors pEAK12d and pDEST12.2

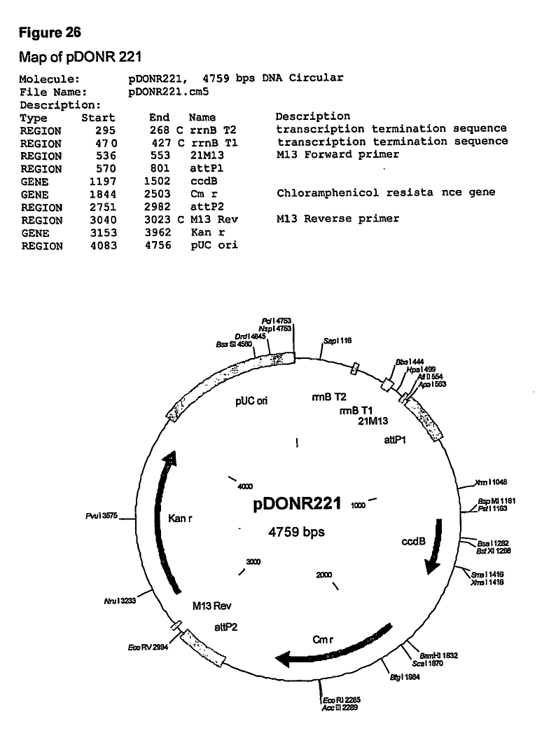

The second stage of the Gateway cloning process involves subcloning of the Gateway modified PCR product into the Gateway entry vector pDONR221 (Invitrogen, FIG. 26) as follows: 5 ∥l of purified product from PCR2 were incubated with 1.5 μl pDONR221 vector (0.1 μg/μl), 2 μl BP buffer and 1.5 μl of BP clonase enzyme mix (Invitrogen) in a final volume of 10 μl at RT for 1 h. The reaction was stopped by addition of proteinase K 1 μl (2 μg/μl ) and incubated at 37° C. for a further 10 min. An aliquot of this reaction (1 μl) was used to transform E. coli DH10B cells by electroporation as follows: a 25 μl aliquot of DH10B electrocompetent cells (Invitrogen) was thawed on ice and 1 μl of the BP reaction mix was added. The mixture was transferred to a chilled 0.1 cm electroporation cuvette and the cells electroporated using a BioRad Gene-Pulser™ according to the manufacturers recommended protocol. SOC media (0.5 ml), which had been pre-warmed to room temperature, was added immediately after electroporation. The mixture was transferred to a 15 ml snap-cap tube and incubated, with shaking (220 rpm) for 1 h at 37° C. Aliquots of the transformation mixture (10 μl and 50 μl) were then plated on L-broth (LB) plates containing kanamycin (40 μg/ml) and incubated overnight at 37° C.

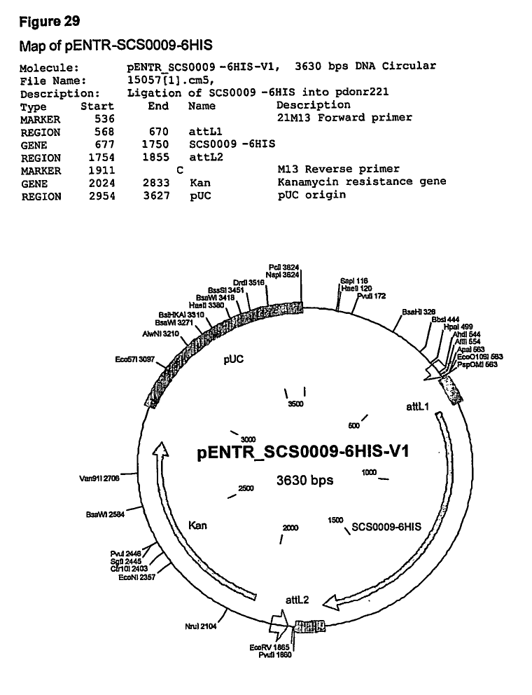

Plasmid mini-prep DNA was prepared from 5 ml cultures from 6 of the resultant colonies using a Qiaprep Turbo 9600 robotic system (Qiagen). Plasmid DNA (150-200 ng) was subjected to DNA sequencing with 21M13, M13Rev and SCS0009-SP1 primers using the BigDyeTerminator system (Applied Biosystems cat. no. 4390246) according to the manufacturer's instructions. The primer sequences are shown in Table 1. Sequencing reactions were purified using Dye-Ex columns (Qiagen) or Montage SEQ 96 cleanup plates (Millipore cat. no. LSKS09624) then analyzed on an Applied Biosystems 3700 sequencer. Plasmid eluate (2 μl or approx. 150 ng) from one of the clones which contained the correct sequence (pENTR-SCS0009-6HIS, plasmid ID 15057, FIG. 29) was then used in a recombination reaction containing 1.5 μl of either pEAK12d vector or pDEST12.2 vector (FIGS. 27 & 28) (0.1 μg/μl), 2 μl LR buffer and 1.5 μl of LR clonase (Invitrogen) in a final volume of 10 μl. The mixture was incubated at RT for 1 h, stopped by addition of proteinase K (2 μg) and incubated at 37° C. for a further 10 min. An aliquot of this reaction (1 μl) was used to transform E. coli DH10B cells by electroporation as follows: a 25 μl aliquot of DH10B electrocompetent cells (Invitrogen) was thawed on ice and 1 μl of the LR reaction mix was added. The mixture was transferred to a chilled 0.1 cm electroporation cuvette and the cells electroporated using a BioRad Gene-Pulser™ according to the manufacturer's recommended protocol. SOC media (0.5 ml), which had been pre-warmed to room temperature, was added immediately after electroporation. The mixture was transferred to a 15 ml snap-cap tube and incubated, with shaking (220 rpm) for 1 h at 37° C. Aliquots of the transformation mixture (10 μl and 50 μl) were then plated on L-broth (LB) plates containing ampicillin (100 μg/ml) and incubated overnight at 37° C.

Plasmid mini-prep DNA was prepared from 5 ml cultures from 6 of the resultant colonies subcloned in each vector using a Qiaprep Turbo 9600 robotic system (Qiagen). Plasmid DNA (200-500 ng) in the pEAK12d vector was subjected to DNA sequencing with pEAK12F, pEAK12R and SCS0009-SP1 primers as described above. Plasmid DNA (200-500 ng) in the pDEST12.2 vector was subjected to DNA sequencing with 21M13, M13Rev and SCS0009-SP1 primers as described above. Primer sequences are shown in Table 1.

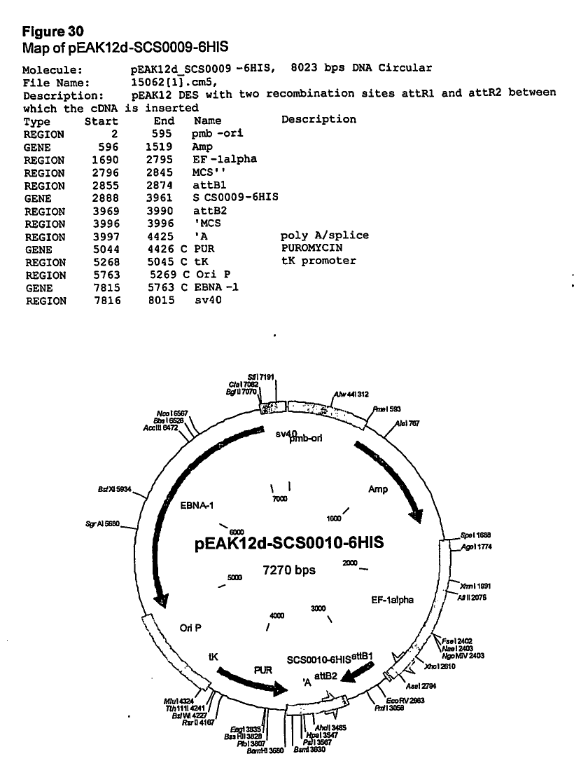

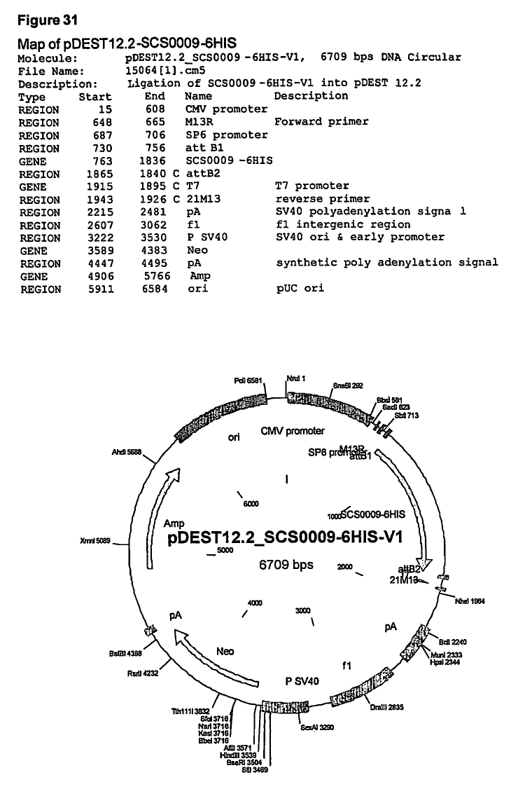

CsCl gradient purified maxi-prep DNA was prepared from a 500 ml culture of one of each of the sequence verified clones (pEAK12d-SCS0009-6HIS, plasmid ID number 15062, FIG. 30, and pDEST12.2-SCS0009-6HIS, plasmid ID 15064, FIG. 31) using the method described by Sambrook J. et al., 1989 (in Molecular Cloning, a Laboratory Manual, 2^{nd} edition, Cold Spring Harbor Laboratory Press). Plasmid DNA was resuspended at a concentration of 1 μg/μl in sterile water (or 10 mM Tris-HCl pH 8.5) and stored at −20° C.

TABLE V
SCS0009 cloning and sequencing primers

| | |
|---|---|
| Primer | Sequence (5′-3′) |

| | |
|---|---|
| SCS0009- | ACC ATG CCC AGC GGC TGC CGC TGC CTG CAT |
| AP1 | CTC G |
| SCS0009- | AGT CAC GCC CAT GGA AGC CTT TGT CAC AGA |
| AP2 | ACT TGC |
| SCS0009- | GCA AGT TCT GTG ACA AAG GCT TCC ATG GGC |
| AP3 | GTG ACT GC |
| SCS0009- | TCA CAG TGC TGT GGT CTT TCC AGG CTC AGG |
| AP4 | GGG CAA GTC |
| SCS0009- | AA GCA GGC TTC GCC ACC ATG CCC AGC GGC |
| EX1 | TGC CGC TG |
| SCS0009- | GTG ATG GTG ATG GTG CAG TGC TGT GGT CTT |
| EX2 | TCC AG |
| GCP | G GGG ACA AGT TTG TAC AAA AAA GCA GGC |
| Forward | TTC GCC ACC |
| GCP | GGG GAC CAC TTT GTA CAA GAA AGC TGG GTT |
| Reverse | TCA ATG GTG ATG GTG ATG GTG |
| SCS0009- | TGA TGC GGC CTT GTG CTA AC |
| SP1 | |
| pEAK12F | GCC AGC TTG GCA CTT GAT GT |
| pEAK12R | GAT GGA GGT GGA CGT GTC AG |
| 21M13 | TGT AAA ACG ACG GCC AGT |
| M13REV | CAG GAA ACA GCT ATG ACC |
| T7 primer | TAA TAC GAC TCA CTA TAG GG |
| SP6 primer | ATT TAG GTG ACA CTA TAG |

Sequence in red = overlap with adjacent exon.
Underlined sequence = Kozak sequence
Bold = Stop codon
Italic sequence = His tag

### Example 4

### Identification and Description of SCS0009 Domains

4.1 Identification

A bioinformatic tool called SMART (http://smart.embl-heidelberg.de/) was used to identify the putative domains of SCS0009 and of the splice variant SCS0009-SV3. Results are shown in FIG. 2. In addition, Prosite was also run on the sequences (http://us.expasy.org/prosite/).

TABLE VI
Domains within the query sequence SCS0009 of 352 residues
Confidently predicted domains, repeats, motifs and features:

| name | begin | end | E-value |
|---|---|---|---|
| signal peptide | 1 | 22 | — |
| EGF | 28 | 58 | 3.16e+01 |
| EGF | 59 | 89 | 7.76e−03 |
| EGF CA | 93 | 141 | 3.51e−02 |
| EGF CA | 143 | 179 | 6.64e−11 |
| EGF CA | 181 | 217 | 9.47e−07 |
| low complexity | 218 | 233 | — |
| transmembrane | 276 | 298 | — |
| low complexity | 306 | 322 | — |

TABLE VII
Domains within the query sequence SCS0009-SV3 of 377 residues
Confidently predicted domains, repeats, motifs and features:

| name | begin | end | E-value |
|---|---|---|---|
| signal peptide | 1 | 22 | — |
| EGF | 28 | 58 | 3.16e+01 |
| EGF | 59 | 90 | 5.04e−02 |
| EGF CA | 89 | 123 | 4.41e−02 |
| EGF CA | 129 | 166 | 3.32e−02 |
| EGF CA | 168 | 204 | 6.64e−11 |
| EGF CA | 206 | 242 | 9.47e−07 |
| low complexity | 243 | 258 | — |
| transmembrane | 301 | 323 | — |
| low complexity | 331 | 347 | — |

TABLE VIII
Domains within the query sequence SCS0009-SV4 of 134 residues
Confidently predicted domains, repeats, motifs and features:

| name | begin | end | E-value |
|---|---|---|---|
| signal peptide | 1 | 22 | — |
| EGF | 28 | 58 | 3.16e+01 |
| EGF | 59 | 89 | 7.76e−03 |
| EGF | 94 | 129 | 1.23e−05 |

Partial ScanProsite results:
SCS0009
>PDOC00016 PS00016 RGD Cell attachment sequence [pattern] [Warning: pattern with a high probability of occurrence].
382-384 RGD

4.2 Description of the Domains
EGF. Epidermal growth factor-like domain. Interpro annotation:

A sequence of about thirty to forty amino-acid residues long found in the sequence of epidermal growth factor (EGF) has been shown MEDLINE:, MEDLINE:88196363, MEDLINE:84117505, MEDLINE:91145344, MEDLINE:85063790, MEDLINE: to be present, in a more or less conserved form, in a large number of other, mostly animal proteins. The list of proteins currently known to contain one or more copies of an EGF-like pattern is large and varied. The functional significance of EGF domains in what appear to be unrelated proteins is not yet clear. However, a common feature is that these repeats are found in the extracellular domain of membrane-bound proteins or in proteins known to be secreted (exception: prostaglandin G/H synthase). The EGF domain includes six cysteine residues which have been shown (in EGF) to be involved in disulphide bonds. The main structure is a two -stranded β-sheet followed by a loop to a C-terminal short two-stranded sheet. Subdomains between the conserved cysteines vary in length.
GF_CA. Calcium-binding EGF-like domain. Interpro annotation:

A sequence of about forty amino-acid residues long found in the sequence of epidermal growth factor (EGF) has been shown to be present in a large number of membrane-bound and extracellular, mostly animal protein s (see IPR000561). Many of these proteins require calcium for their biological function and a calcium-binding site has been found to be located at the N-terminus of some EGF-like domains. Calcium-binding may be crucial for numerous protein-protein interactions. For human coagulation factor IX it has been shown that the calcium-ligands form a pentagonal bipyramid. The first, third and fourth conserved negatively charged or polar residues are side chain ligands. Latter is possibly hydroxylated (see IPR000152). A conserved aromatic residue as well as the second conserved negative residue are thought to be involved in stabilizing the calcium-binding site. Like in non-calcium binding EGF-like domains there are six conserved cysteines and the structure of both types is very similar as calcium-binding induces only strictly local structural changes. 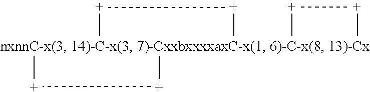
‘n’: negatively charged or polar residue [DEQN]
‘b’: possibly beta-hydroxylated residue [DN]
‘a’: aromatic amino acid
‘C’: cysteine, involved in disulfide bond
‘x’: any amino acid.
PS00016; RGD

The sequence Arg-Gly-Asp, found in fibronectin, is crucial for its interaction with its cell surface receptor, an integrin. What has been called the ‘RGD’ tripeptide is also found in the sequences of a number of other proteins, where it has been shown to play a role in cell adhesion. These proteins are: some forms of collagens, fibrinogen, vitronectin, von Willebrand factor (VWF), snake disintegrins, and slime mold discoidins. The ‘RGD’ tripeptide is also found in other proteins where it may also, but not always, serve the same purpose.

4.3 Conclusion

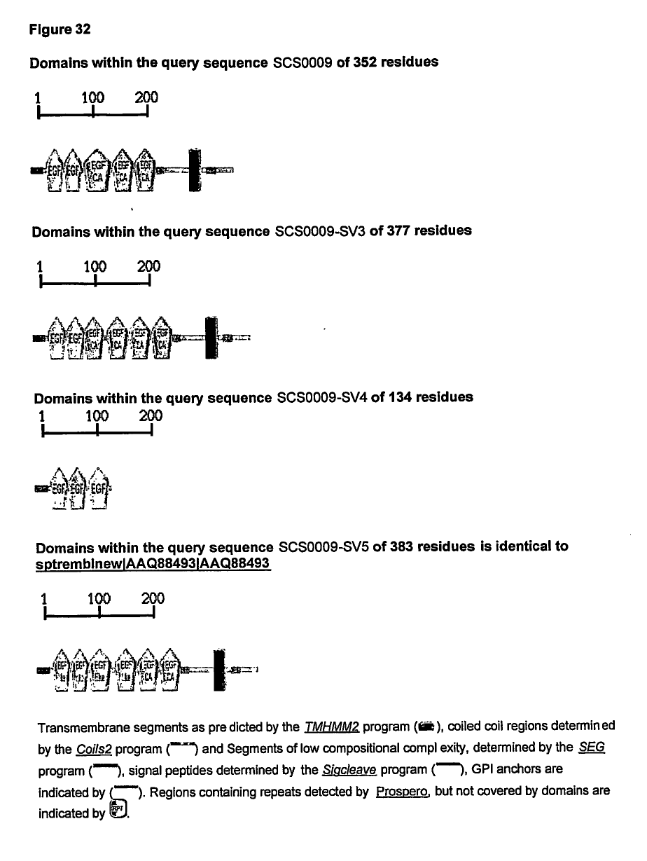

In addition to the comments put forward in example 1, it is clear, based on SMART results (FIG. 32), that SCS0009, SCS0009-SV3 and SCS0009-SV5 have a common domain organization. However, SCS0009, but not SCS0009-SV3 nor SCS0009-SV5, contains an RGD tripeptide sequence, indicating that SCS0009 may interact with integrin cell surface receptors, which are involved in cell adhesion. SCS0009 and SCS0009-SV3 contain also a dileucine, an ER membrane retention signal (KKXX-like motif in the C-terminus: KTTA), as well as a probable zinc finger of C3HC4 type (identified by PSORT: http://psort.nibb.ac.jp/form2.html, results not shown). As such, SCS0009 shows distinct features in its signaling pathway compared with SCS0009-SV3 and SCS0009-SV5. Among the splice variants described, SCS0009-SV4 does not contain a transmembrane domain. As such, it could represent a secreted protein, which may act as integral SCS0009 antagonist in vivo. In addition, when exposed to the action of an unknown protease, DLK1 generates a soluble peptide referred to as Fetal antigen 1 (FA1) (Jensen et al.). It is therefore probable that soluble SCS0009 and SCS0009-SV3 (as well as for other splice variants of SCS0009) can be generated through the action of FA1. These soluble SCS0009 polypeptides may also act as integral SCS0009 antagonist in vivo and might show particularly useful in the diagnosis and treatment of diseases, as described in therapeutic uses above.

### Example 5

### Metabolic Endocrinology Assays Suitable for Exploration of the Biological Relevance of Proteins Function

A number of metabolic endocrinology-related assays have been developed by the Applicant and are of use in the investigation of the biological relevance of protein function. Examples of metabolic endocrinology-related assays that have been developed by the Applicant include four cell-based assays for metabolic endocrinology. These are discussed below.

5.1 Differentiation to adipocyte assay

Inhibition of adipocyte differentiation is an in vitro model for reduction of adipose mass believed to be important in reducing insulin resistance in diseases such as diabetes and Polycystic Ovary Syndrome (PCOS). The goal is to identify protein(s) that inhibit differentiation of pre-adipocytes to adipocytes. The 3T3-L1 mouse preadipocyte cell line is induced to differentiate to adipocytes with insulin+IBMX. The finding that differentiation is inhibited by TNFα+cyclohexamide is used as a positive control.

5.2 Tritiated glucose uptake (3T3 L1)

The goal Is to identify protein(s) that stimulate glucose uptake as a model for insulin-resistance in adipose during diabetes or PCOS. Adipocytes used are mouse 3T3-L1 preadipocytes that have been differentiated.

5.3 Tritiated glucose uptake (primary human adipocytes)

The goal is to identify protein(s) that stimulate glucose uptake as a model for insulin-resistance in adipose during diabetes or PCOS. Primary human adipocytes are used.

5.4 Tritiated glucose update (primary human skeletal muscle cells)

The goal is to identify protein(s) that stimulate glucose uptake as a model for insulin-resistance in muscle tissue during diabetes or PCOS. Primary human skeletal muscle cells are differentiated into myotubes and then used in the assay.

### REFERENCES

Andersen D C and Krummen L, Curr Opin Biotechnol, 13: 117-23, 2002.
Baker K N et al., Trends Biotechnol, 20: 149-56, 2002.
Blagoev B and Pandey A, Trends Biochem Sci, 26: 639 -41, 2001.
Bock A, Science, 292: 453-4, 2001.
Bunz F, Curr Opin Oncol, 14: 73-8, 2002.
Burgess R R and Thompson N E, Curr Opin Biotechnol, 12: 450-4, 2001.
Chambers S P, Drug Disc Today, 14: 759-765, 2002.
Chu L and Robinson D K, Curr Opin Biotechnol, 13: 304-8, 2001.
Cleland J L et al., Curr Opin Biotechnol, 12: 212-9, 2001.
Coleman R A et al., Drug Discov Today, 6: 1116-1126, 2001.
Constans A, The Scientist, 16(4): 37, 2002.
Davis B G and Robinson M A, Curr Opin Drug Discov Devel, 5: 279-88,2002.
Dougherty D A, Curr Opin Chem Biol, 4: 645-52,2000.
Garnett M C, Adv Drug Deliv Rev, 53: 171-216, 2001.
Gavilondo J V and Larrick J W, Biotechniques, 29: 128-136, 2000.
Gendel S M, Ann N Y Acad Sci, 964: 87-98, 2002.
Giddings G, Curr Opin Biotechnol, 12: 450-4,2001.
Golebiowski A et al., Curr Opin Drug Discov Devel, 4: 428-34, 2001.
Gupta P et al., Drug Discov Today, 7: 569-579, 2002.
Haupt K, Nat Biotechnol, 20 : 884-885, 2002.
Hruby V J and Balse P M, Curr Med Chem, 7: 945-70, 2000.
Johnson D E and Wolfgang G H, Drug Discov Today, 5: 445-454, 2000.
Kane J F, Curr Opin Biotechnol, 6: 494-500, 1995.
Kolb A F, Cloning Stem Cells, 4: 65-80, 2002.
Kuroiwa Y et al., Nat Biotechnol, 20: 889-94, 2002.
Lin Cereghino G P et al., Curr Opin Biotechnol, 13: 329-332, 2001.
Lowe C R et al., J Biochem Biophys Methods, 49: 561-74, 2001.
Luo B and Prestwich G D, Exp Opin Ther Patents, 11: 1395-1410, 2001.
Mulder N J and Apweiler R, Genome Biol, 3(1):REVIEWS2001, 2002
Nilsson J et al., Protein Expr Purif, 11: 1-16, 1997.
Pearson W R and Miller W, Methods Enzymol, 210: 575-601, 1992.
Pellois J P et al., Nat Biotechnol, 20: 922-6, 2002.
Pillai O and Panchagnula R, Cur Opin Chem Biol, 5: 447-451, 2001
Rehm B H, Appl Microbiol Biotechnol, 57: 579-92, 2001.
Robinson C R, Nat Biotechnol, 20: 879-880, 2002.
Rogov S I and Nekrasov A N, Protein Eng, 14: 459-463, 2001.
Schellekens H, Nat Rev Drug Discov, 1: 457 -62, 2002
Sheibani N, Prep Biochem Biotechnol, 29: 77-90, 1999.
Stevanovic S, Nat Rev Cancer, 2: 514-20, 2002.
Templin M F et al., Trends Biotechnol, 20: 160-6, 2002.
Tribbick G, J Immunol Methods, 267: 27-35, 2002.
van den Burg B and Eijsink V, Curr Opin Biotechnol, 13: 333-337, 2002.
van Dijk M A and van de Winkel J G, Curr Opin Chem Biol, 5: 368-74, 2001.
Villain M et al., Chem Biol, 8: 673-9,2001.

1-41. (canceled)

42. A composition of matter comprising:
a) an isolated polypeptide having Preadipocyte-Factor-1-like activity selected from the group consisting of: 1) the amino acid sequence recited in SEQ ID NO: 2; 2) the mature form of the polypeptide whose sequence is recited in SEQ ID NO: 2 (SEQ ID NO:4); 3) active variants of the amino acid sequence of SEQ ID NO: 2, wherein any amino acid specified in the chosen sequence is non-conservatively substituted, provided that no more than 15% of the amino acid residues in the sequence are so changed; and 4) the active fragment, precursor, salt, or derivative of the amino acid sequences given in 1), 2), or 3);
b) an isolated polypeptide that is a naturally occurring allelic variant of the sequence given by SEQ ID NO: 2 or SEQ ID NO:4;
c) an isolated polypeptide that is a naturally occurring allelic variant of the sequence given by SEQ ID NO: 2 or SEQ ID NO:4, wherein the variant is the translation of a single nucleotide polymorphism;
d) a polypeptide as set forth in a) or b) or c), wherein the polypeptide binds specifically an antibody or a binding protein generated against SEQ ID NO: 2 or SEQ ID NO:4 or a fragment thereof;
e) a fusion protein comprising a polypeptide as set forth in a) or b) or c) or d);
f) an antagonist of a polypeptide as set forth in a) or b) or c) or d), wherein said antagonist comprises an amino acid sequence resulting from the non-conservative substitution, the deletion or both the non-conservative substitution and deletion of one or more residues into the corresponding polypeptide;
g) a ligand which binds specifically to a polypeptide a) or b) or c) or d);
h) a polypeptide a) or b) or c) or d) or e), wherein said polypeptides are in the form of active conjugates or complexes with a molecule chosen from radioactive labels, fluorescent labels, biotin, or cytotoxic agents;
i) a peptide mimetic designed on the sequence or the structure or the sequence and structure of a polypeptide as set forth in a) or b) or c) or d);
j) an isolated nucleic acid encoding for an isolated polypeptide selected from the group consisting of:
1) polypeptides as set forth in a) or b) or c) or d);
2) a fusion protein comprising a polypeptide as set forth in a) or b) or c) or d); or
3) an antagonist of a polypeptide as set forth in a) or b) or c) or d), wherein said antagonist comprises an amino acid sequence resulting from the non-conservative substitution, the deletion or both the non-conservative substitution and deletion of one or more residues into the corresponding polypeptide;
k) an isolated nucleic acid sequence comprising SEQ ID NO: 1; nucleotides 122-1180 of SEQ ID NO: 1; or a complement of either sequence;
l) a purified nucleic acid which:
1) hybridizes under high stringency conditions; or
2) exhibits at least about 85% identity over a stretch of at least about 30 nucleotides with a nucleic acid selected from the group consisting of SEQ ID NO: 1; nucleotides 122-1180 of SEQ ID NO: 1; or a complement of either sequence;
m) a vector comprising a nucleic acid as set forth in j) or k) or l);
n) a polypeptide encoded by the nucleic acid of j) or k) or l);
o) a host cell comprising a vector or nucleic acid as set forth in j) or k) or l) or m);
p) a transgenic animal cell comprising a vector or nucleic acid as set forth in j) or k) or l) or m) and having enhanced or reduced expression levels of a polypeptide as set forth in a) or b) or c) or d);
q) a transgenic non-human animal that has been transformed to have enhanced or reduced expression levels of a polypeptide as set forth in a) or b) or c) or d);
r) a compound that enhances the expression level of a polypeptide as set forth in a) or b) or c) or d) in a cell or animal; or
s) a compound that reduces the expression level of a polypeptide as set forth in a) or b) or c) or d) in a cell or animal.

43. The composition of matter according to claim 42, wherein said composition of matter comprises a polypeptide, peptide mimetic, nucleic acid, cell, or compound that enhances or reduces the expression of a polypeptide and a pharmaceutically acceptable carrier.

44. The composition of matter according to claim 42, wherein the fusion protein further comprises one or more amino acid sequence selected from the protein sequences: membrane-bound protein, immunoglobulin constant region, multimerization domains, extracellular proteins, signal peptide-containing proteins, or export signal-containing proteins.

45. The composition of matter according to claim 42, wherein the ligand antagonizes or inhibits the Preadipocyte-Factor-1-like activity of a polypeptide.

46. The composition of matter according to claim 45, wherein the ligand is a monoclonal antibody, a polyclonal antibody, a humanized antibody, an antigen binding fragment, or the extracellular domain of a membrane-bound protein.

47. The composition of matter according to claim 42, wherein said vector comprises a nucleic acid molecule that is operatively linked to expression control sequences allowing expression in prokaryotic or eukaryotic host cells of the encoded polypeptide.

48. A method for determining the activity and/or the presence of a Preadipocyte-Factor-1-like polypeptide in a sample comprising:
a) providing a protein-containing sample;
b) contacting said sample with a ligand that specifically binds to a polypeptide comprising:
1) an isolated polypeptide having Preadipocyte-Factor-1-like activity selected from the group consisting of: i) the amino acid sequence recited in SEQ ID NO: 2; ii) the mature form of the polypeptide whose sequence is recited in SEQ ID NO: 2 (SEQ ID NO:4); iii) active variants of the amino acid sequence of SEQ ID NO: 2, wherein any amino acid specified in the chosen sequence is non-conservatively substituted, provided that no more than 15% of the amino acid residues in the sequence are so changed; and iv) the active fragment, precursor, salt, or derivative of the amino acid sequences given in i), ii), or iii);
2) an isolated polypeptide that is a naturally occurring allelic variant of the sequence given by SEQ ID NO: 2 or SEQ ID NO:4;
3) an isolated polypeptide that is a naturally occurring allelic variant of the sequence given by SEQ ID NO: 2 or SEQ ID NO:4, wherein the variant is the translation of a single nucleotide polymorphism; or
4) a polypeptide as set forth in a) or b) or c), wherein the polypeptide binds specifically an antibody or a binding protein generated against SEQ ID NO: 2 or SEQ ID NO:4 or a fragment thereof; and
c) determining the presence or said ligand bound to said polypeptide.

49. A method of using the composition of claim 42 for a) producing cells capable of expressing a polypeptide; b) making a polypeptide; c) the treatment of a disease; d) the preparation of pharmaceutical compositions; e) the screening candidate compounds; f) identifying a candidate compound; or g) determining the presence or the amount of a transcript or of a nucleic acid.

50. The method according to claim 49, wherein said method comprises genetically engineering cells with a vector or a nucleic acid comprising:
a) an isolated nucleic acid encoding for an isolated polypeptide selected from the group consisting of:
1) an isolated polypeptide having Preadipocyte-Factor-1-like activity selected from the group consisting of: i) the amino acid sequence recited in SEQ ID NO: 2; ii) the mature form of the polypeptide whose sequence is recited in SEQ ID NO: 2 (SEQ ID NO:4); iii) active variants of the amino acid sequence of SEQ ID NO: 2, wherein any amino acid specified in the chosen sequence is non-conservatively substituted, provided that no more than 15% of the amino acid residues in the sequence are so changed; and iv) the active fragment, precursor, salt, or derivative of the amino acid sequences given in i), ii), or iii);
2) an isolated polypeptide that is a naturally occurring allelic variant of the sequence given by SEQ ID NO: 2 or SEQ ID NO:4;
3) an isolated polypeptide that is a naturally occurring allelic variant of the sequence given by SEQ ID NO: 2 or SEQ ID NO:4, wherein the variant is the translation of a single nucleotide polymorphism;
4) a polypeptide as set forth in 1) or 2) or 3), wherein the polypeptide binds specifically an antibody or a binding protein generated against SEQ ID NO: 2 or SEQ ID NO:4 or a fragment thereof; or
5) a fusion protein comprising a polypeptide as set forth in 1) or 2) or 3) or 4);
b) an isolated nucleic acid sequence comprising SEQ ID NO: 1; nucleotides 122-1180 of SEQ ID NO: 1; or a complement of either sequence;
c) a purified nucleic acid which:
1) hybridizes under high stringency conditions; or
2) exhibits at least about 85% identity over a stretch of at least about 30 nucleotides with a nucleic acid selected from the group consisting of SEQ ID NO: 1; nucleotides 122-1180 of SEQ ID NO: 1; or a complement of either sequence; or
d) a vector comprising a nucleic acid as set forth in a) or b) or c).

51. The method according to claim 49, wherein said method comprises a method for making a polypeptide comprising culturing a transformed host cell under conditions in which the nucleic acid or vector is expressed, and recovering the polypeptide encoded by said nucleic acid or vector from the culture.

52. The method according to claim 49, wherein said method comprises the treatment of a disease needing an increase in the Preadipocyte-Factor- 1 -like activity that comprises the administration of a therapeutically effective amount of a polypeptide, a peptide mimetic, a nucleic acid, a cell, or a compound as set forth in claim 42.

53. The method according to claim 49, wherein said method of treatment comprises the administration of a therapeutically effective amount of an antagonist, a ligand, or of a compound as set forth in claim 42.

54. The method according to claim 49, wherein said method for screening candidate compounds effective to treat a disease related to the Preadipocyte-Factor-1-like polypeptides comprises:
a) contacting a cell, a transgenic animal cell, or a transgenic non-human animal having enhanced or reduced expression levels of the Preadipocyte-Factor-1-like polypeptide, with a candidate compound; and
b) determining the effect of the compound on the animal or on the cell.

55. The method according to claim 49, wherein said method for identifying a candidate compound as an antagonist/inhibitor or agonist/activator of a Preadipocyte-Factor-1-like polypeptide comprises:
a) contacting said polypeptide, said compound, and a mammalian cell or a mammalian cell membrane capable of binding the polypeptide; and
b) measuring whether the molecule blocks or enhances the interaction of the polypeptide, or the response that results from such interaction, with the mammalian cell or the mammalian cell membrane.

56. The method according to claim 49, wherein said method for determining the presence or the amount of a transcript or of a nucleic acid encoding a Preadipocyte-Factor-1-like polypeptide comprises:
a) providing a nucleic acids-containing sample;
b) contacting said sample with a nucleic acid as set forth in claim 42; and
c) determining the hybridization of said nucleic acid with a nucleic acid into the sample.
